(19) United States
(12) Patent Application Publication
(10) Pub. No.: US 2026/0283860 A1
(43) Pub. Date: Sep. 24, 2026

(54) ABSORBENT ARTICLE COMPRISING AN OUTER COVER AND A DETACHABLE INSERT

(71) Applicant: Essity Hygiene and Health Aktiebolag, Göteborg (SE)

(72) Inventors: Annika HERMENIUS, Göteborg (SE); Sofia RICHARDSON, Göteborg (SE); Lina AUGUSTSSON, Göteborg (SE); Verica TERENTIC, Göteborg (SE)

(73) Assignee: Essity Hygiene and Health Aktiebolag, Göteborg (SE)

(21) Appl. No.: 19/166,068

(22) PCT Filed: Mar. 20, 2023
(86) PCT No.: PCT/EP2023/057066

§ 371 Date: Sep. 17, 2025

(51) Int. Cl. A61F 13/49; A61F 13/15; A61F 13/505; A61F 13/53

(52) U.S. Cl. CPC A61F 13/49006; A61F 13/505; A61F 13/53; A61F 2013/15121; A61F 2013/5055; A61F 2013/530481

(57) ABSTRACT

An absorbent article includes front, back, and crotch sections, with a reusable outer cover including a garment portion and an absorbent insert. In at least one of the front and back sections, the outer cover includes at least one flap member extending from the garment portion with an outward facing inner side facing the inward facing side of the insert in a front or back edge portion. At least one of the front and back sections includes a set of one or more attachment areas where a surface area of the inward facing side of the insert is detachably attached to a surface area of the outward facing inner side via at least one surface fastening material, such that the front or back edge portion of the insert is detachably attached to the outer cover. The inward facing side of the insert may form the innermost side of the article.

### FIELD OF THE INVENTION

The present invention relates to an absorbent article comprising an outer cover and a detachable insert. The present invention further relates to an outer cover. Also, the present invention relates to an insert for use with an outer cover. Additionally, the present invention relates to a package enclosing a plurality of inserts.

### BACKGROUND

Absorbent articles, in the forms of diapers and incontinence pads, are generally used for babies and incontinent adults for the absorption of bodily exudates, such as blood, urine, sweat and feces. In particular when looking at products that are classified as heavy incontinence articles for adults, which articles have a relatively high absorption capacity, these products are often used by physically and/or mentally impaired persons. Generally, these heavy incontinence products will be handled by a caregiver to put on and remove from the wearer's body and bought by care homes, hospitals and/or care giving relatives.

Requirements put on these products may include: being leakproof, being easy to use and change, being comfortable to wear, do not negatively affect the wearer's skin health, and maintain the wearer's dignity during changing and wearing.

To meet these requirements, currently used incontinence products are still mostly disposable products, with single-use products being the standard for heavy incontinence. Disposable products are easy to use, since they require very limited handling after use. Both the production and disposal of these products, however, result in a lot of material waste. After use, the products either end up as land fill, or, in particular for adults who are on one or more heavy medications, the absorbent articles may be incinerated.

Currently, most disposable products are made of, or contain a substantial amount of materials that are derived from petroleum, such as polypropylene and/or polyethylene, appearing in the form of cloth-like nonwoven web materials and films. Through the increasing awareness that petroleum has a limited availability and causes various pollution problems both during product manufacturing and products being discarded, there is also an increasing need for more sustainable alternatives. To this end, products have been proposed which comprises an outer chassis and an absorbent insert, wherein one or both of the outer chassis and the absorbent insert is/are reusable. For this type of products, it is of importance to ensure that the absorbent insert is correctly positioned in relation to the wearer, such that the security against leakage and the desired comfort is maintained during use of the product.

Thus, it is an aim to provide an absorbent article that may be a sustainable alternative to current disposable products.

Further, it is an aim to provide an absorbent article that may satisfactory fulfil the above-mentioned requirements and/or provide an improvement in relation to current products.

### SUMMARY

### Definitions

With “wearer” is meant a person which may be wearing an absorbent article as described herein.
With “user” is meant a person handling the absorbent article as described herein, i.e. preparing an absorbent article for use and/or applying the absorbent article to the wearer. The user may be a wearer or a caregiver to the wearer.
With “liquid permeable” is meant herein a material being liquid permeable as understood in the sense of the art of absorbent articles. As such, the material is not intended to hinder fluids from leaking through the material to any significant degree. Known liquid permeable materials are for example such as being used as topsheet materials in conventional disposable absorbent products. In another example, conventional textile materials such as woven or knitted materials are generally liquid permeable. A liquid permeable material may for example be a textile material being free from any liquid resistant coatings or treatments. A liquid permeable material may for example be a textile material which do not absorb fluid to any significant degree.
With “breathable” is meant herein a material allowing water vapor to escape through the material.

For example, materials which are breathable while still being liquid-impermeable are commonly used in absorbent articles as backsheet materials. Such backsheets materials may be breathable while still preventing liquids from passing through the backsheet material. Such breathable, liquid impermeable materials may for example be porous polymeric films, and laminates from porous polymeric films and nonwovens.

Textile materials which form apertures, such as knitted or woven textile materials, are generally breathable.

The breathability of a material is normally measured by the water vapor transmission rate. For example, a breathable material suitable for absorbent articles may have a water vapor transmission rate of more than 1000 g/ms/24 h at 37.8 degrees Celsius according to ASTEM; E398.
With “total absorption capacity” is meant herein the total absorption capacity, as measured according to the Rothwell method, ISO 11948.
With “use cycle” of an absorbent article is meant herein a series of subsequent steps comprising the application of the absorbent article to a wearer, the wearer wearing the article for a limited period of time (for example during one or a few urinary discharges), and the removal of the absorbent article from the wearer.
With “reusable” is meant herein an item which intended to be used for more than one use cycle. Thus, a reusable item is an item which may be used throughout a repeated number of use cycles. If required, the reusable item may be cleaned, for example washed, or otherwise restored between use cycles.

For example, a reusable item may be designed to be used for at least 50 use cycles, such as at least 100 use cycles, or at least 500 use cycles.
With “single-use” is meant herein an item which is intended to be used for only one use cycle, and then discarded. Thus, an item being configured for single-use is typically disposed after being used by a wearer for a limited period of time (for example during one or a few urinary discharges).

1By “washable” it is meant that the outer cover may be cleaned by laundering. The outer cover may thus be subjected to an aqueous solution containing detergent without losing its structural features. This aqueous solution may be heated as part of the laundering process, e.g. to 40° C. or 60° C.

A washable outer cover should be able to be washed at least a predetermined number of times, without significantly deteriorated features. In particular, the washable outer cover should be able to be washed a predetermined number of times without significant impairment of the fastening members, such as any insert fastener members or of fastening members for fastening the outer cover around the waist of the wearer. Also, the washable outer cover should preferably be able to be washed a predetermined number of times without significant impairment of the elasticity of the leg elastics and/or other materials, such as fabrics, of the outer cover. Finally, the washable outer cover should preferably be able to be washed a predetermined number of times without significant impairment of the colour of the outer cover.

For example, it is preferred herein that the washable outer cover is able to sustain at least 20 washes in 60° C., and yet more preferred that the washable outer cover is able to sustain at least 50 washes in 60° C.

Also, it is preferred that the washable outer cover is “machine washable”, i.e. that the washes mentioned in the above may take place in a washing machine.
With “stretchable” is meant herein a material which may be stretched from an original measure and are able recover to essentially the same measure, when subject to forces during normal use of the article.
With “surface fastening material” is meant herein a material which may be substantially uniformly distributed over the surface of an area, so as provide for substantially uniform fastening by said surface fastening material over the area. Thus, any portion of the area being provided with the surface fastening material will be ready for attachment via the surface fastening material.

In some examples, a surface fastening material may provide for continuous fastening over the surface of the area comprising the surface fastening material. Such a surface fastening material may for example be an adhesive, which may be applied over the area.

In some examples, a surface fastening material may provide for fastening in a plurality of discrete fastening elements, the density of the fastening elements being such that substantially uniform fastening is provided over the area comprising the surface fastening material. For example, a density of such discrete fastening elements may be at least 10 discrete fastening elements per cm2, or more preferred at least 50 discrete fastening elements per cm2.

For example, the plurality of discrete fastening elements may provide for mechanical and/or frictional fastening over the area comprising the surface fastening material. Examples of surface fastening materials comprising discrete fastening elements may be hook materials and/or loop materials for forming hook/hook type or hook/loop type connections.
With a “fastener area comprising a surface fastening material” is meant a surface area over which a surface fastening material is substantially uniformly distributed.
With “detachable” fastening is meant herein that the fastenier areas may be used to attach and detach a corresponding fastener area of an insert multiple times without substantial impairment of their function. For example, for any reusable fastener area, the fastener area may provide for at least 50 cycles of attachment/detachment, such as at least 150 times or at least 300 times
“Knitting” is a method of constructing a fabric by interlocking a series of loops of one or more yarns. There are two major classes of knitting namely warp and weft knitting.
“Weaving” is a method or process of interlacing two yarns of similar materials so that they cross each other at right angles to produce woven fabric. The warp yarns run lengthwise in the fabric and the filling threads or picks, run from side to side.

Jacquard is a system of weaving or knitting that utilizes a versatile pattern mechanism to produce intricate designs by using punch cards controlling the patterns produced. Jacquard knit may be valid for circular, flatbed, warp or weft knit.

In the context of the present disclosure, a “nonwoven” is a manufactured sheet, web or batt of directionally or randomly orientated fibers, bonded by friction, and/or cohesion and/or adhesion, excluding paper and products which are woven, knitted, tufted, stitch-bonded incorporating binding yarns or filaments, or felted by wet-milling, whether or not additionally needled. The fibers may be of natural or man-made origin and may be staple or continuous filaments or be formed in situ. Commercially available fibers have diameters ranging from less than about 0.001 mm to more than about 0.2 mm and they come in several different forms: short fibers, continuous single fibers, untwisted bundles of continuous filaments, and twisted bundles of continuous filaments.

Nonwoven fabrics can be formed by many processes such as meltblowing, spunbonding, solvent spinning, electrospinning, and carding.
The term “nonwoven”, mentioned in relation to the different parts of the insert, which in term of their properties are located between the groups of paper and cardboard on the one hand and textiles on the other hand. As regards nonwovens, a large number of extremely varied production processes are used, such as airlaid, wetlaid, spunlaced, spunbond, meltblown techniques etc. The fibres may be in the form of endless fibres or fibres prefabricated with an endless length, as synthetic fibres produced in situ or in the form of staple fibres. Alternatively, they may be made from natural fibres or from blends of synthetic fibres and natural fibres.

In the context of the present disclosure, a longitudinal length is to be understood as the longest length of a specified part or sub part in the longitudinal direction. Analogously, a transversal width is to be understood as the widest width of a specified part or sub part in the transversal direction.

When referring to the length of a set comprising one or more items (i.e. a length spanned by a set comprising one or more items), it is referred to the maximum longitudinal length including all of the items in the set, i.e. regardless of whether the items are transversally aligned or not.

Similarly, when referring to the width of a set comprising one or more items (i.e. a width spanned by a set comprising one or more items), it is referred to the maximum transversal width including all of the items in the set, i.e. regardless of whether the items are longitudinally aligned or not.

By the term “entirely contained within” is meant herein that an item being “entirely contained within” e.g. a part or portion does not extend beyond, e.g. does not protrude from, the part or portion.

For the purposes of the present disclosure, and unless otherwise explicitly mentioned, lengths, widths and other dimensions of a disposable absorbent hygiene product are measured with the disposable absorbent hygiene product in a flattened-out state on a flat surface with any contracting elastics being deactivated. With “deactivated” is meant that the elastics are not active to contract any parts to which it is attached. To deactivate the elastics, the elastics may for example be cut at one or more locations, or the elastics may be removed. Optionally, in some instances, it may be possible to stretch out the elastics such that the portions to which the elastics are arranged are completely flattened out. The measures obtained in such a stretched out state may correspond to the measures as obtained if the elastics were deactivated.

When the length of an active part of an elastic element along another portion is to be determined, the start and end point of the active part of the elastic element may initially be marked on the relevant portion. Then, the elastic elements may be deactivated, and the distance between the marked start and end point may be measured on the relevant portion when in a flattened-out state.

The active part of the elastic element is the part which, in use, may be active to contract any part to which it is attached. As such, using the procedure in the above, parts of an elastic element which cannot be activated, such as glued down end sections of elastic threads, are disregarded. As used herein, the term “area” of a portion of a product corresponds to the surface area of a flat surface covered by the portion when the product is placed on the flat surface in a flattened out state with any elastics removed or deactivated.

To determine the flat area of an item such as the outer cover, which may comprise several joined panels or sections, it may be necessary to cut the item into several flat pieces to determine the area of each piece.

N. B. herein, when it is referred to the area of the outer cover, or the length of the outer cover, or other dimensions of the outer cover, it is referred to the measures as found when any flap members of the outer cover is/are in their use position.

### 1.Absorbent Article

In a first aspect, the present disclosure relates to an absorbent article according to claim 1. Thus, there is provided an absorbent article extending along a longitudinal direction, and along a transversal direction, which is perpendicular to the longitudinal direction, and wherein a thickness direction is defined perpendicular to both the longitudinal and transversal directions, said thickness direction being an inward direction towards the wearer when the article is worn, and an outward direction away from the wearer when the article is worn. The absorbent article comprises a front section, a back section and a crotch section extending between the front section and the back section along the longitudinal direction.

Further, the absorbent article comprises
a reusable outer cover, comprising a garment portion that extends at least in the front section, and the back section, said front and back sections of the garment portion being adapted to fit around a wearer's waist, with the front section being positioned at or close to the wearer's belly button and the back section being positioned against the wearer's lower back when the article is worn; and
an absorbent insert comprising a liquid impermeable backsheet, a liquid permeable topsheet, and an absorbent assembly provided in between said backsheet and said topsheet, the insert being shaped to, in use, cover a wearer's pubic area and a predefined part of the buttocks with an inward facing side comprising the topsheet directed towards the wearer, and an outward facing side directed towards the garment portion of the outer cover. In at least one, preferably each, of the front section and the back section of the absorbent article, the outer cover comprises a flap member extending from said garment portion with an outward facing inner side of the flap member facing the inward facing side of the insert in a front or back edge portion, respectively, of the insert.

In said at least one, preferably each, of the front section and the back section of the absorbent article, the absorbent article comprises a set of one or more attachment areas, wherein, in each attachment area, a surface area of the inward facing side of the insert is detachably attached to a surface area of said outward facing inner side of the flap member via at least one surface fastening material, such that the front or back edge portion, respectively, of the insert is detachably attached to the outer cover in said set of attachment areas while, in at least said crotch section of the absorbent article, the inward facing side of the insert forms the innermost side of the absorbent article, so as to be directly adjacent the wearer's skin, when the article is in use.

In the absorbent article, the leakage protection and absorptive capacity is mainly provided by the absorbent insert, while the reusable outer cover is adapted to hold the insert in its place on the wearer's lower torso. At least a portion of the inward facing side of the insert will be directly adjacent the wearer's skin and thus available for receiving bodily exudates from a wearer.

Through the outer cover and insert being detachable from one-another, the reusable outer cover may be used multiple times with fresh inserts.

### Properties of the Absorbent Article

The outer cover is reusable, i.e. intended to be used for more than one use cycle. If required, the reusable outer cover may be cleaned, for example washed, or otherwise restored when necessary. For example, the reusable outer cover could be used for a number of use cycles, each use cycle involving use of a fresh insert, before becoming soiled such that the reusable outer cover requires cleaning before being used for new use cycles.

Thus, for example, the outer cover may be washable.

The absorbent insert may also be at least partly reusable, for example washable. However, the absorbent insert will usually be soiled after each use cycle. As such, the absorbent insert may be cleaned, for example washed, or otherwise restored after each use cycle.

However, preferably, the absorbent insert may be adapted for single use, i.e. intended to be used for one use cycle only and then discarded. As such, the insert is adapted to be used for a limited time, such as for receiving only one or a few bodily discharges. After use, the absorbent insert adapted for single use is disposed.

As such, the absorbent article comprising the reusable outer cover and the absorbent insert presents advantages over prior art single use absorbent articles, in that sustainability is improved by at least a part of the absorbent article being reusable.

The absorbent article has a longitudinal length extending along a longitudinal direction from a front edge to a back edge of the absorbent article via the crotch section. The longitudinal length may be adapted such that the absorbent article is sized to fit adults.

Optionally, the absorbent article has a total absorption capacity of at least 1000 ml, such as from 1000 to 4000 ml. As such, the article has an absorption capacity intended for adult wearers.

Optionally, the article has a total absorption capacity, and the insert providing at least 90%, such as at least 95% or at least 99 % of the total absorption capacity of the absorbent article. Thus, as mentioned in the above, the absorption capacity of the absorbent article is provided essentially by the absorbent insert, whereas the outer cover is relatively non-absorbing and serves the function to hold the insert in a correct position relative to the wearer's body.

### Breathable Outer Cover & Insert

As mentioned in the above, the leakage protection and absorption capacity of the absorbent article is primarily provided by the absorbent insert, while the reusable outer cover is adapted to hold the insert in its place on the wearer's lower torso.

Further, the liquid impermeable backsheet of the absorbent insert provides for a majority of the required liquid impermeability of the absorbent article. As such, the outer cover may be mainly liquid permeable, e.g. made by liquid permeable materials such as textile materials.

For example, at least 50%, such as at least 70% or at least 90 %, of a total area of the outer cover may be liquid permeable.

Since the outer cover is not required to be liquid impermeable, the outer cover may be adapted in view of other, desired properties. For example, the outer cover may be made by materials having properties being advantageous for the skin health of the wearer. For example, the outer cover may be made by materials providing a desired breathability, softness and/or stretchability. Suitable materials may for example be found among textile materials.

Thus, preferably, the outer cover may be mainly breathable.

For example, at least 50% of a total area of the outer cover, such as at least 70%, or at least 90% of a total area of the outer cover may be breathable.

Thus, a majority of the area of the outer cover may be made by a liquid permeable, breathable material to the benefit of the skin health of the wearer. Preferably, the liquid permeable, breathable material may also be washable.

As mentioned in the above, the absorbent insert comprises a liquid impermeable backsheet. As such, the outward facing side of the absorbent insert may comprise the liquid impermeable backsheet. Optionally, the outward facing side of the absorbent insert may consist of the liquid impermeable backsheet.

The backsheet of the insert may be breathable, to the benefit of the skin health of the user.

As such, the absorbent article may comprise an outer cover which is mainly breathable as set out in the above, and an absorbent insert having a liquid impermeable backsheet which is breathable, such that the absorbent article as a whole is also mainly breathable.

The one or more garment portion(s) that forms the outer cover may comprise one garment portion forming the front section, the back section, and a crotch section extending longitudinally between the front section and the back section, and the garment portion forms a pair of leg edges, each leg edge facing towards a respective leg opening when the article is worn. Thus, the garment portion of the outer cover may extend over the crotch section of the absorbent article, interconnecting the front section and the back section of the outer cover. As such, the garment portion may be adapted to fit around a wearer's waist with the front section being positioned at or close to the wearer's belly button, the back section being positioned against the wearer's lower back, and the crotch section being positioned between two leg openings formed by the outer cover.

Accordingly, the outer cover may substantially entirely cover the insert, when the outer cover and the insert are used together to form the absorbent article. Thus, the outer cover forms the entire outward facing surface, or garment facing side, of the absorbent article, substantially hiding the insert from view. As a result, the absorbent article has an appearance that is closer to regular underpants when being worn, which will be perceived by wearers are more dignified and comfortable than wearing the currently used disposable diapers.

Moreover, the outer cover may protect the insert by providing an additional layer in the outward direction of the insert, such that any tearing or friction from the outside, as could result from the movements of the wearer, will initially be taken up by the outer cover rather than by the outward facing side, e.g. the backsheet, of the insert. Thus, the insert may stay more securely in place so as to perform its function.

The outer cover may comprise a pair of leg elastic members, arranged along the leg edges of the garment portion, at least in the crotch section thereof.

The leg elastic members contribute to the correct positioning of the absorbent article on the wearer, adding to the capability of the outer cover to hold the absorbent insert in a correct position visavi the wearer to take up bodily discharges. As such, the leg elastic members contribute to controlling the position of the crotch portion of the absorbent insert when attached to the outer cover. Moreover, the leg elastic members contribute to the comfort and fit of the outer cover, providing an undergarment-like appearance to the absorbent article.

### Attachment Between the Insert and the Outer Cover in the Absorbent Article

As set out in the above, in the absorbent article proposed herein, the set of one or more attachment areas is such that attachment between the insert and the outer cover is made via the inward facing side of said insert. This contributes to providing a secure bond between the insert and the outer cover. As such, as seen from the outside direction when the article is worn, the attachment area is covered both by the insert itself and by an outward facing layer of the outer cover. Accordingly, the attachment area is difficult to reach by curious and plucking fingers of the wearer. Moreover, the garment portion of the outer cover forming the outward facing surface of the absorbent article also at the location of the attachment area, can move relatively independently from the insert without directly impacting the attachment area. Thus, movements of the wearer, which may cause the outwardly directed side of the absorbent article to be pulled or rubbed, are less likely to negatively impact the attachment of the insert to the outer cover.

An attachment area of the absorbent article is an area in which a surface area of the inward facing side of the insert is detachably attached to a surface area of said outward facing side of the flap member via at least one surface fastening material.

As such, it is to be understood that the surface fastening material is distributed over the entire attachment area—that is over a surface area of the inward facing side of the insert and/or the corresponding surface area of the outward facing side of the flap member.

Accordingly, the attachment between the insert and the outer cover takes place over the entire attachment area, and thus any forces to which the attachment area is subject when the absorbent article is in use, is distributed over the attachment area. This provides for advantageous distribution of load between the insert and the outer cover, avoiding peak loads as could appear e.g. when using point connections such as buttons or snaps. Further, attachment via a surface fastening material provided in the areas may be user friendly, in that attachment between the inner facing side of the insert and the outer facing side of the outer cover may be accomplished while giving some tolerance for the exact relative position between the insert and the outer cover—again, this is in contrast to point connections such as buttons or snaps. Further, as will be explained more in the below, attachment using one or more surface fastening materials may be accomplished using relatively thin and flexible surface fastening materials, contributing to the comfort of the wearer and avoiding risks for chafing.

Thus, by definition, the set of one or more attachment areas, comprises all attachment aeras in the front or back section, respectively, wherein a surface area of the inward facing side of the insert is detachably attached to a surface area of the outward facing inner side of the flap member via at least one surface fastening material.

Possibly, the absorbent article could provide for additional detachable attachment between the outer cover and the insert, in addition to the set of attachment areas providing detachable attachment between a surface area of the inward facing side of the insert and the outward facing inner side of the flap member. For example, an additional attachment could be provided between the outward facing side of the insert and the garment portion of the outer cover. However, any such additional attachment should preferably be minimised, since the additional attachment may impair the benefits obtained by using the set of one or more attachment areas as proposed herein.

Rather, in the at least one, preferably each, of the front section and the back section of the absorbent article, the set of one or more attachment areas providing detachable attachment between a surface area of the inward facing side of the insert and the outward facing inner side of the flap member may constitute the only attachment between the insert and the outer cover.

Preferably, each of the front section and the back section of the absorbent article comprises a set of attachment areas providing detachable attachment between a surface area of the inward facing side of the insert and the outward facing inner side of the flap member, wherein said sets of attachment areas constitute the only attachment between the insert and the outer cover in the absorbent article.

As such, in said at least one, preferably each, of the front section and the back section of the absorbent article, the outward facing inner side of the flap member of the outer cover may comprise a set of one or more insert fastener areas, each insert fastener area comprising a surface fastening material for providing detachable attachment to the inward facing surface of the insert at least in at least one out of said set of one or more attachment areas. The insert fastener area is thus the area over which the surface fastening material is distributed.

Alternatively, or in addition, in said at least one, preferably each, of the front section and the back section of the absorbent article, the inward facing side of the insert may comprise a set of one or more cover fastener areas, each cover fastener area comprising a surface fastening material for providing detachable attachment to the outward facing surface of said flap member at least in at least one of said set of one or more attachment areas. The cover fastener area is thus the area over which the surface fastening material is distributed.

Preferably, the outward facing side of the flap member of the outer cover comprises a set of one or more insert fastener areas, each insert fastener area comprising a surface fastening material, and the inward facing side of the insert comprises a set of one or more cover fastener areas, wherein the surface fastening material of the set of insert fastener areas and the surface fastening material of the set of cover fastener areas are corresponding surface fastening materials for forming said detachable connection. Accordingly, said set of attachment areas are formed where said set of one or more insert fastener areas overlap with said set of one or more cover fastener areas.

As mentioned in the above, each insert fastener area of the outer cover may comprise a surface fastening material. In this case, for example, the surface fastening material could provide detachable attachment to a material layer on the inward facing side of the insert, which material layer is directly attachable to the surface fastening material of the insert fastener areas. For example, such a material layer could be formed by a topsheet material or a leakage barrier forming material of the insert. As such, the insert fastener area could attach to any position on the material layer, such as to any position on the inward facing side of the insert, or to any position on the topsheet.

Similarly, each cover fastener area of the insert could comprise a surface fastening material for providing detachable attachment to any position on the outward facing side of the flap member of the outer cover.

However, preferably, as mentioned in the above, the insert comprises a set of one or more cover fastener areas comprising a surface fastening material, and the outer cover comprises a set of one or more insert fastener areas comprising a corresponding surface fastening material, for detachably attaching said insert to said outer cover.

As such, preferably, the insert comprises a set of one or more cover fastener areas comprising a surface fastening material being different from the remainder of the material layer or layers of the inward facing side of the insert. For example, the cover fastener areas may comprise a surface fastening material being different from the topsheet material layer and/or a leakage barrier forming layer on the inward facing side of the insert. As such, the attachment area may be adapted to provide for possible connection between the insert and the outer cover only at the cover fastener areas. Thus, the connection between the outer cover and the insert may be controlled as regards for example the location of the connection and/or the strength of the connection formed.

For example, the insert fastener areas of the outer cover may be adapted so as to provide for a stronger connection when attached to the cover fastener areas, than when attached to any other portion of the inward facing side of the insert.

For example, each of the insert fastener areas of the outer cover may be adapted so as to be non-adherened to the inward facing side of the insert apart from at the cover fastener areas. This may facilitate handling and ensure that the insert is properly positioned in relation to the outer cover.

Similarly, in some embodiments, depending on the design of the one or more flap members, it may be preferred that the set of insert fastener areas of the flap member cover less than the total area of the outward surface of the flap member, so as to control the location of the connection between the outer cover and the insert.

Preferably, the one or more cover fastener areas of the insert comprises a first surface fastening material, and the one or more insert fastener areas of the outer cover comprises a second surface fastening material, cooperative with the first surface fastening material, for forming a detachable connection.

For example, each one of the set of one or more cover fastener areas of the insert may have an approximately similar size and shape as a corresponding one of the set of one or more inert fastener areas.

### Detachable Attachment

With “detachable” fastening is meant herein that the attachment area may be used for attachment and detachment multiple times without substantial impairment of its function.

For example, the attachment area may provide for at least 50 cycles of attachment/detachment, such as at least 150 cycles or at least 500 cycles.

It is to be understood that if the absorbent insert is a single-use insert, to provide detachable fastening of the insert to the outer cover for a number of use cycles may imply fastening a new insert to the outer cover for each use cycle.

Hence, for a single-use insert, it may be sufficient that the one ore more cover fastener areas are adapted to be detachable from the corresponding one or more insert fastener areas for a relatively lower number of use cycles, such as for example for at least 10 use cycles. Still, when used with a re-usable outer cover, the one or more insert fastener areas may be adapted to be detachable from the corresponding one or more insert fastener areas (from a number of individual single-use inserts) for a relatively larger number of use cycles, such as for example for at least 50 use cycles, or for at least 150 or at least 500 use cycles.

Hence, it may be desired that the one or more cover fastener areas of the insert are detachable from a corresponding one or more insert fastener areas on the outer cover without impairing the function of the reusable one or more insert fastener areas of the outer cover.

For example, the one or more cover fastener areas may be non-disintegrating fastener areas, i.e. the cover fastener areas may be such that they do not disintegrate when being removed from attachment with an insert fastener area.

For example, it may be desired that the one or more cover fastener areas of the insert are detachable from a corresponding one or more insert fastener areas of the outer cover without leaving residues such as debris on the reusable one or more insert fastener areas of the outer cover. If residues of a disintegrated cover fastener area of an insert is left on an insert fastener area of an outer cover, this might impair the function of insert fastener area when it is desired to perform a subsequent attachment of a new insert to the outer cover.

For example, surface fastening materials such as adhesives may be prone to leave residues behind when removed from an attachment.

For example, residues of adhesive left on an insert fastener area of an outer cover might impair the function of a subsequent attachment to the insert fastener area, and/or the residues may be difficult to remove from the insert fastener area by washing.

Thus, the surface fastening material of each attachment area in the set of one or more attachment areas may preferably be a non-adhesive surface fastening material. (Non-adhesive meaning that the surface fastening material is not an adhesive or glue.)

Similarly, the surface fastening material of each insert fastener area in the set of one or more insert fastener areas of the outer cover may preferably be a non-adhesive surface fastening material.

Similarly, the surface fastening material of each cover fastener area in the set of one or more cover fastener areas of the insert may preferably be a non-adhesive surface fastening material.

For example, each of the at least one surface fastening materials of the set of one or more attachment areas of the absorbent article may provide detachable attachment through mechanical fastening and/or frictional fastening.

For example, in at least one, preferably each, of the insert fastener areas in the set of one or more insert fastener areas of the outer cover, the surface fastening material may be adapted for allowing detachable mechanical and/or frictional fastening thereto by a matching fastening material.

For example, in at least one, preferably each, of the set of one or more cover fastener areas of the insert, the surface fastening material may be adapted for allowing detachable mechanical and/or frictional fastening thereto by a matching fastening material.

That the fastening material is adapted for allowing mechanical and/or frictional fastening thereto implies that it may provide for detachable connection through a relatively large number of use cycles. Further, mechanical and/or frictional fastening may be provided which sufficiently retains its function also through a number of wash cycles.

For example, a surface fastening material adapted for allowing detachable mechanical and/or frictional fastening thereto by mechanical and/or frictional fastening may be comprise a plurality of discrete fastening elements. For example, a density of such discrete fastening elements may be at least 10 discrete fastening elements per cm2, more preferred at least 50 discrete fastening elements per cm2.

For example, a surface fastening material adapted for allowing detachable mechanical and/or frictional fastening thereto by a matching fastening material may be a fastening material comprising a plurality of discrete fastening elements comprising stems which project from the upper surface of the fastener area over which it is distributed.

For example, the discrete fastening elements comprising stems which project may be in the form of hooks. A surface fastening material comprising hooks may for example be adapted for allowing detachable mechanical and/or frictional fastening to another surface material comprising hooks, i.e. forming a hook/hook connection. In another example, a surface fastening material comprising hooks may be adapted for allowing detachable mechanical and/or frictional fastening to another surface fastening material comprising loops, i.e. forming a hook/loop connection.

With a “hook” it is intended to mean the discrete, projecting fastening element. The hooks can have any shape. Examples of hooks comprise pins, for example straight pins, angled pins, curved pins, tapered pins, limbed or multi-limbed pins, hooks, limbed or multi-limbed hooks, mushroom shaped protrusions, and palm tree shaped protrusions. The hooks may have any type of cross-section such as round, oval, square, rectangular, or polygonal. The hooks may have a solid core. Within one surface fastening material, all hooks may have the same shape. Alternatively, one surface fastening material may comprise several different shapes of hooks.

In surface fastening material comprising “loops”, the discrete fastening elements may be e.g. in the form of fibrous loops.

The surface fastening material may be arranged immediately on an underlying surface, such as e.g. a surface of the outward facing side of a flap member of the outer cover or on the inward facing side of the insert.

For example, the surface fastening material may be applied immediately on the outward facing side of the flap member, when in a use position. For example, the surface fastening material may comprise a plurality of discrete fastening elements, such as for example hooks, which project directly from the outward facing side of the flap member.

By selecting surface fastening materials as proposed herein, the cover fastener areas and/or the insert fastener areas, as well as the attachment areas, may be flexible, i.e. that they may have sufficient flexibility to be able to adequately follow the wearer's body without creating unnecessary chafing or discomfort.

Also, by selecting surface fastening materials as proposed herein, the cover fastener areas and/or the insert fastener areas, as well as the attachment areas, may be relatively thin, again so as to avoid unnecessary chafing of discomfort.

For example, optionally a height of the attachment area, when in an attached state, is less than 3 mm, such as less than 2 mm.

For example, when the fastening material comprises a plurality of discrete fastening elements, such as discrete fastening elements comprising stems which project in the outward direction, a height of said discrete fastening elements may be less than 3 mm, such as less than 2 mm, or less than 1.5 mm.

For example, when the outer cover comprises one or more insert fastener members (as will be described in the below), each insert fastener member may have a height of less than 3 mm, such as less than 2 mm, or less than 1.5 mm.

For example, when the insert comprises one or more cover fastener members (as will be described in the below), each cover fastener member may have a height of less than 3 mm, such as less than 2 mm, or less than 1.5 mm.

### Fastener Members

Optionally, the insert comprises one or more cover fastener members arranged on the inward facing side of the insert, each cover fastener member providing at least one out of said one or more cover fastener areas in said set.

The one or more cover fastener members may for example be attached to the inward facing side of the insert by means of adhesive or welds. For example, ultrasonic welds or heat-sealing welds may be used.

Alternatively, or in addition, the outer cover comprises one or more insert fastener members arranged on the outward facing side of said flap member, each insert fastener member providing at least one of the insert fastener areas in said set.

The one or more insert fastener members may for example be attached to the outward facing side of the respective flap member by means of seams or welds.

Thus, the surface fastening material may be arranged on the cover fastener member or insert fastener member, respectively. The surface fastening material may be arranged to form one or more areas on a cover fastener member or insert fastener member, such corresponding to the one or more cover fastener areas or insert fastener areas provided by the cover fastener member or insert fastener member, respectively.

To this end, the cover fastener member or the insert fastener member may comprise a sheet formed base

For example, at least one, such as all, of the one or more insert fastener members may comprise a sheet formed base having a length in the longitudinal direction and a width in the transversal direction, and comprising the one or more areas of a surface fastening material.

For example, at least one, such as all, of the one or more cover fastener members may comprise a sheet formed base having a length in the longitudinal direction and a width in the transversal direction, and comprising one or more cover fastener areas of a surface fastening material.

The sheet formed base of the insert fastener member/cover fastener member may have generally parallel first and second surfaces, with the first surface being attached to the outward facing side of the flap member/inward facing side of the insert.

As such, an insert fastener member or cover fastener member may comprise a sheet formed base comprising one or more insert fastener areas or one or more cover fastener areas, respectively.

Preferably, at least one, such as all, of the one or more insert fastener members or cover fastener members may comprise a sheet formed base comprising no more than one insert fastener area or cover fastener member, respectively.

For example, the surface fastening material may comprise a plurality of discrete fastening elements comprising stems which project from a surface of the sheet formed base. The stems may for example be hooks which are adapted to be attached to corresponding loops.

### Cover Fastener Areas and Insert Fastener Areas

It may be preferred that any surface fastening material comprising stems which projects are arranged on the outer cover, i.e. in the insert fastener areas. This is because the arrangement with the insert fastener areas applied on the outward facing side of the flap members of the outer cover hinders the projections from coming into contact with the wearer's skin.

Thus, for example, for at least one, such as all, of the insert fastener areas of said set, the surface fastening material may comprise a plurality of discrete fastening elements, such as discrete fastening elements comprising stems which project in the outward direction, as seen when the flap member is in the use position.

It may be preferred that any surface fastening material on the inward facing side of the insert is free from projections. As such it may be ensured, that even if incorrectly fastened to the outer cover, such that some part of a cover fastener area is left uncovered by the flap member, the cover fastener area should not risk damaging the wearer's skin.

For example, at least one, such as all, of the one or more cover fastener members in said set may comprise a loop material.

### Insert Fastener Areas of Outer Cover

For example, as mentioned in the above the one or more insert fastener areas may comprise projecting members such as hooks. For example, one or more insert fastener member may comprise a hook material. Through the positioning of the insert fastener areas on the outward facing side of the flap member, such hooks are thus directed away from the wearer's skin during use, lowering any risk of causing frictional damage to a wearer's skin during use.

Further, it has been realised that hook materials are generally more able than loop materials to retain their properties through a number of wash cycles. As such, for a washable outer cover, by providing insert fastener areas comprising a plurality of discrete fastening elements comprising stems which project from a surface, such as for example hooks, on the outward facing side of the flap member, the risk of the projections coming in contact with the wearer's skin is reduced while the function of the insert fastener area throughout the lifetime of a washable outer cover is ensured.

As mentioned in the above, the one or more insert fastener areas may be adapted to attach onto any surface on the wearer facing inward facing side of the insert, such as to attach onto a nonwoven surface, such as a topsheet surface of the insert. However, it has been found that there may be a risk of debris from e.g. nonwoven surface such as from a topsheet material loosen from the insert as the insert is removed from the insert fastener areas. Such debris may gather on the insert fastening aera, gradually impairing its function. Further, such debris has been found to be difficult to remove from the fastener areas, even when washed.

Accordingly, it may be preferred that the one or more insert fastener areas are adapted to detachably attach onto corresponding one or more designated cover fastener areas comprising a corresponding surface fastening material and arranged on the inward facing side of the insert.

Further, correct positioning of the insert in relation to the outer cover may be better ensured when the attachment of the insert is made via designated cover fastener areas of the insert.

Optionally, the garment portion of the outer cover and the set of insert fastener areas of the outer cover are non-adherend to one another.

As such, the insert fastener areas and the material of the garment portion, e.g. the fabric panels of the garment portion, may be selected such that the insert fastener areas are non-adherend to the garment portion. Thus, the risk of the insert fastener areas unintentionally fastening onto the garment portion is removed.

Additionally, the flap member and the one or more insert fastener areas may be non-adherend to one another.

As mentioned in the above, the flap member and the garment portion may comprise the same material, for example a fabric material being non-adherend to the insert fastener areas.

The same may be applied to any further fastening members of the outer cover.

For example, the back section of the outer cover may comprise one or more first waist attachment means, and the front section of the outer cover may comprise one or more second waist attachment means, corresponding to the first waist attachment means, so as to provide for detachable fastening of the outer cover around the waist of a wearer. For example, such waist attachment means may comprise hook and/or loop materials. As such, the first and/or second waist attachment means may be non-adherend to the garment portion and/or to the flap member.

### Cover Fastener Areas of Insert

For example, at least one, such as all, of the one or more cover fastener areas may be formed by a material layer of loop-material.

Loop material may be relatively soft to the touch, such that, even if the material were to accidentally be placed against a wearer's skin, the risk of causing frictional damage is relatively low.

Further, a loop material may be made by materials being less expensive and/or more advantageous in terms of environmental sustainability than a corresponding hook material. As such, it is advantageous to be able to provide the loop material on a single-use insert to be used with a hook material on a reusable outer cover.

It is to be understood, that the features and explanations in the above relating to fastening materials, attachment areas, cover fastener areas and insert fastener areas apply equally to the absorbent article of the first aspect as described herein, the outer cover of the second aspect as described in the below, and the insert of the third aspect as described in the below.

### Flap Members of the Absorbent Article

Optionally, the flap member has a use position wherein a space is formed between the outward facing inner side of the flap member and the inward facing side of the garment portion, which space is open in a longitudinal direction towards the crotch section for receiving the respective back edge portion or front edge portion, respectively, of the insert therein, while the insert extends over the crotch portion of the absorbent article.

The flap member extends from the garment portion in the back section or in the front section, respectively. For example, the flap member may be attached to an inward facing surface of the garment portion in the back section and/or in the front section, respectively.

Optionally, the insert has an insert longitudinal length, and in said at least one, preferably each, out of the back section and the front section, each flap member has a longitudinal flap length being such that, in the use position and when the insert is attached to the outer cover, the flap member extends over less than 20% of the insert longitudinal length, such as over less than 15% of the insert longitudinal length, preferably over less than 10% of the insert longitudinal length.

As such, each flap member covers a limited length of the insert longitudinal length, leaving the majority of the inward facing side of the insert comprising the topsheet exposed such that at least a portion of the topsheet is directly adjacent the wearer's skin.

For example, the portion of the topsheet being directly adjacent the wearer's skin may be a main portion of the topsheet. As such, the main portion of the topsheet being directly adjacent the wearer's skin may have a longitudinal length of at least 50% of a total topsheet length, such as at least 75% of a total topsheet length or at least 80% of a total topsheet length.

The total topsheet length may be substantially equal to the insert longitudinal length, i.e. the topsheet may extend over the full length of the insert.

As such, for example, when the outer cover comprises at least one front flap member in the front section, and at least one back flap member in the back section, the front and the back flap members may have respective longitudinal lengths being such that, in the use position and when the insert is attached to the outer cover, the front and the back flap members together extend over less than 50% of the insert longitudinal length. For example, the front and the back flap members may together extend over less than 75% of the insert longitudinal length, such as less than 80% of the insert longitudinal length.

Optionally, the insert has an insert area, and, in said at least one, preferably each, out of the back section and the front section, a flap area of the outward facing inner side of each one of the flap member of the outer cover is such that, in the use position and when the insert is attached to the outer cover, the flap member extends over less than 20% of the insert area, such as over less than 15% of the insert area, or preferably over less than 10% of the insert area.

As such, for example, when the outer cover comprises at least one flap member in each of the front and back sections of the outer cover, the one or more front flap member(s) and the one or more back flap member(s) may have respective areas being such that, in the use position and when the insert is attached to the outer cover, the one or more front flap member(s) and the one or more back flap member(s) together extend over less than 50% of the insert area. For example, all of the flap members of the absorbent article may together extend over less than 75% of the insert area, such as less than 80% of the insert area.

In said at least one, preferably each, of the front section (Sf) and the back section (Sb) of the absorbent article, the set of one or more attachment areas spans over a transversal fastening width. The transversal fastening width may preferably be no less than 70% of a maximum width of the insert (200) in said front edge portion or back edge portion, respectively. For example, the transversal fastening width may be no less than 80% or no less than 90% of a maximum width of the insert in said front edge portion or back edge portion, respectively.

### Subsets of Attachment Areas of the Absorbent Article

Optionally, in said at least one, preferably each, out of the back section and the front section, the set of one or more attachment areas comprises a first subset of one or more attachment areas, and a second subset of one or more attachment areas, where the first and second subsets are spaced around the longitudinal central axis of the insert. Preferably, the first and second subsets are symmetrically spaced around the longitudinal axis of the insert.

Accordingly, the absorbent article may be free from attachment areas between the outer cover and the insert over a spacing width as measured transversely between said first and second subsets of attachment areas.

This contributes to the shaping of the insert of the absorbent article when the absorbent article is in use. Notably, the front and/or back section of the insert may be held out over the transversal fastening width, so as to secure the position of the insert visavi the wearer, while allowing for relative movement between the outer cover and the insert over the spacing width. This allows for the insert to shape in relation to the body of the wearer, for example, to assume a slight cup-shape.

For example, the transversal spacing width may be at least 40% of the transversal fastening width, such as at least 50% or at least 60% of the transversal fastening width.

### Insert Leg Elastic Members of the Absorbent Article

Optionally, the insert extends longitudinally between a front edge and a back edge, and comprises longitudinal side edges connecting the front edge and the back edge. Further, the insert may comprise a pair of insert leg elastic members extending essentially along the longitudinal direction of the insert, and arranged on both sides of the insert, in between the absorbent assembly and the longitudinal side edges of the insert. For each of the front and back section of the absorbent article, a leg elastics width is defined as the maximum transversal width over the insert leg elastic members in said front or back section, respectively. Optionally, the leg elastics width in the front and back section of the absorbent article may be the same, and correspond to a maximum transversal width over the insert leg elastic members of the insert.

The insert leg elastic members contribute to forming the insert around the user's body, and to provide sufficient fluid tightness at the leg openings of the absorbent article when attached to a wearer. As mentioned in the above, also the outer cover may comprise leg elastic members. The insert may be adapted to the outer cover such that, in use, the pair of insert leg elastic members of the insert extend transversally inwards of the pair of leg elastic members of the outer cover. As such, the leg elastic members of the outer cover contribute to providing correct positioning of the insert and the insert leg elastic members, whereas the insert leg elastic members in addition provides for sufficient fluid tightness at the leg openings of the absorbent article to diminish the risk of leakage.

Optionally, in said at least one, preferably each, out of the back section and the front section, the transversal fastening width spanned by the attachment area is no less than the leg elastics width in the front or back section, respectively. The transversal fastening width being no less than the leg elastics width implies that the insert leg elastics may be stretched out in a transversal direction when the insert is attached to the outer cover, thus ensuring proper function and positioning of the insert leg elastics.

Optionally, in said at least one, preferably each, out of the back section and the front section of the absorbent article, the insert comprises a pair of leg edge portions, each leg edge portion extending longitudinally along a respective insert leg elastic member, and transversally between the insert leg elastic member and a longitudinal side edge of the insert so as to define a maximum transversal leg edge width between an insert leg elastic member and a respective longitudinal side edge, the maximum transversal leg edge width being at least 1 cm, such as at least 2 cm. For example, the maximum transversal leg edge width may be no more than 4 cm.

Along an insert leg elastic member, the transversal leg edge width is the distance measured parallel to the transversal direction from the longitudinal side edge to the portion of the insert leg elastic member located closest to the side edge. As such, the transversal leg edge width may vary along the leg elastic member. The maximum transversal leg edge width is the maximum distance measured parallel to the transversal direction from the longitudinal side edge to the portion of the leg elastic member located closest to the side edge, along the insert leg elastic member.

Herein, a maximum transversal leg edge width is defined in each of the front section and the back section of the absorbent article. Optionally, the maximum transversal leg edge width may be the same in the front section and in the back section of the absorbent article, and may correspond to a maximum transversal leg edge width of the insert.

The pair of leg edge portions extending transversally between the insert leg elastic members and a respective longitudinal side edge of the insert provides for additional leakage security of the absorbent article. In use, the leg edge portions are to extend transversally outwards from the insert leg openings as formed along the insert leg elastic members of the insert.

When combined with the transversal fastening width spanned by the attachment area being no less than the leg elastics width, as set out in the above, the leg edge portions may be at least partially extended in a transversal direction when the insert is attached to the outer cover. As such, the leg edge portions may be automatically folded out to a correct position when the insert is attached to the outer cover. Thus, the attachment area contributes to folding out and holding the leg edge portion in a transversally extending state during fastening of the absorbent article to a wearer, and during use thereof. Accordingly, manual manoeuvres otherwise required for folding out the leg edge portion may be dispensed with. This is a benefit in particular for adult wearers, improving the dignity of the wearer in the care situation by avoiding manual manipulation of leg edge portions located at the crotch region of the wearer.

For example, the transversal fastening width may span into at least 50% of the transversal width of each leg portion, such as at least 75% of the transversal width of each leg portion, or at least 80% of the transversal width of each leg portion.

The pair of insert leg elastic members may further extend along a leg elastics length. In said least one, preferably each, out of the back section and the front section, each attachment area may be positioned longitudinally between an end of the insert leg elastic member and the front edge or back edge respectively, of the insert. As such, the function of the attachment areas to ensure extension of the insert leg elastic members in the longitudinal direction may be improved. For example, the set of one or more cover fastener areas may be confined to a longitudinal portion of the insert not spanned by the leg elastics length. In other words, the set of one or more cover fastener areas is positioned longitudinally between a front end of the insert leg elastic members and a front edge of the insert, or between a back end of the insert leg elastic members and a back edge of the insert, respectively.

As such, when the transversal fastening width is greater than the leg elastics width, and each attachment area is positioned longitudinally in a portion of the insert between the front/back end of the insert leg elastic members and the front/back edge of the insert, the attachment area may ensure stretching in transversal and longitudinal directions of the insert leg elastic members.

The inward facing side of the insert may comprises a pair of inner side leakage barriers, extending in the longitudinal direction of the insert.

An insert comprising a pair of inner side leakage barriers as exemplified in the above and a pair of insert leg elastic members as exemplified in the above will provide double barriers against leakage of bodily fluids from the insert when in use. Side leakage barriers, also known as “standing gathers” are described in more detail in the below in relation to the insert. The side leakage barriers may be stretched out and raised above the inward facing surface of the topsheet.

As such, the proposed positions of the attachment areas as set out in the above has the additional advantage that the positions contribute to raising the side leakage barriers and keeping them in position, when the absorbent article is in use.

In particular, when the insert comprises a pair of insert leg elastic members as described herein, and a pair of inner side leakage barriers as defined herein, the proposed positions of the attachment areas as explained herein being useful to stretch out the insert leg elastics, at the same time further contribute to raising the inner side leakage barriers.

Further features and advantages of the absorbent article will be apparent from the below description of an outer cover for forming an absorbent article, and of an insert for forming an absorbent article. It is to be understood that the features described for the outer cover and the features described for the insert are applicable to the absorbent article as described in the above, alone or in combination.

With the longitudinal length of the absorbent article is meant the maximum longitudinal length from the front edge to the back edge of the absorbent article, i.e. of the outer cover with the insert detachably attached to it, and as measured via the crotch section absorbent article. In other words, the longitudinal length is the maximum longitudinal length as seen over the crotch width Wc of the absorbent article. As such, portions of the absorbent article extending transversally beyond the crotch width may be disregarded when determining the longitudinal length of the absorbent article. For example, transversally extending side portions provided to provide fastening of the absorbent article around the waist of the wearer may be disregarded. The crotch width of the absorbent article is the minimum width of the crotch section of the absorbent article, i.e. the minimum width of the portion of the absorbent article intended to be positioned between the leg openings formed by the absorbent article when in use.

Moreover, it should be understood that the longitudinal length of the absorbent article may deviate from the longitudinal length of the outer cover, since, for example, when the insert is attached to the outer cover, the outer cover may be wrinkled in the longitudinal direction.

### 2. Outer Cover

In a second aspect, the disclosure relates to an outer cover according to claim 28.

As such, the disclosure relates to a reusable outer cover for an absorbent article, the outer cover extending along a longitudinal direction, and along a transversal direction, perpendicular to the longitudinal direction, wherein an inward facing direction is defined being towards the wearer when the article is worn, and an outward facing direction is defined being away from the wearer when the article is worn, the inward facing and outward facing directions being perpendicular to the transversal and longitudinal directions. The outer cover has a longitudinal length that extends along the longitudinal direction and is subdivided into a front section, a back section and a crotch section. The outer cover comprises a garment portion that extends in the front section, the back section, and the crotch portion, said front and back sections of the garment portion being adapted to fit around a wearer's waist, with the front section being positioned at or close to the wearer's belly button, the back section being positioned against the wearer's lower back, and the crotch section being positioned between two leg openings formed by the outer cover, such that the garment portion defines an inward facing surface facing in a direction towards the wearer, and an outward facing surface, facing away from the wearer, when the outer cover is worn.

In at least one, preferably each, of the front section and the back section, the outer cover comprises at least one flap member extending from the garment portion, each flap member having a use position wherein an outward facing inner side of the flap member is facing the inward facing side of the garment portion so as to form a space between the outward facing inner side of the flap member and the inward facing side of the garment portion, which space is open in a longitudinal direction towards the crotch section. Further, in the at least one, preferably each of, the front section and the back section, the outer cover comprises a set of one or more insert fastener areas being positioned on the outward facing inner side of the at least one flap member, each insert fastener area in said set comprising a surface fastening material for detachably attaching an insert to said outer cover, such that, when an insert is attached to the outer cover, said one or more insert fastener areas of the at least one flap member allow detachable attachment to the inward facing side of a respective front edge portion or back edge portion of the insert received in said space, while the insert extends inwardly over at least part of the crotch portion of the garment portion.

As such, the garment portion of the outer cover may be adapted to fit around a wearer's waist with the front section being positioned at or close to the wearer's belly, the back section being positioned against the wearer's lower back, and the crotch section being positioned between two leg openings formed by the outer cover.

Accordingly, the outer cover may substantially entirely cover the insert, when used together in the absorbent article. Thus, the outer cover forms the entire outward facing surface, or garment facing side, of the absorbent article, substantially hiding the insert from view. As a result, the absorbent article has an appearance that is closer to regular underpants when being worn, which will be perceived by wearers are more dignified and comfortable than wearing disposable diapers.

Moreover, the outer cover may protect the insert and provide an additional layer, such that any tearing or friction from the outside, as could result from the movements of the wearer, will initially be taken up by the outer cover. Thus, the insert may stay more securely in place so as to perform its function.

Since the garment portion of the outer cover extends over the crotch portion of the outer cover, any friction or tearing to which the outer cover may be subject when worn, affects the garment portion of the outer cover rather than the absorbent insert attached to the outer cover. Thus, the position of the absorbent insert visavi the wearer may be secured, to the benefit of providing sufficient absorption and hindering leakage.

### Portions of the Outer Cover

The garment portion of the outer cover may comprise a main body, forming a transversally central portion of the garment portion, and extending longitudinally from a back end of the outer cover, between the wearer's legs, and to a front end of the outer cover, when the outer cover is in use. As such, the crotch section of the outer cover, at least a central portion of the front section, and at least a central portion of the back section of the outer cover may be formed by the main body.

In the front and/or back section of the outer cover, the main body may be provided with one or more transversally extending side portions, for providing attachment of the outer cover around the waist of a wearer. For example, a pair of side portions may be arranged in the front section of the outer cover, to be fastened to another pair of side portions arranged in the back section of the outer cover. In other examples, a pair of side portions is arranged in one out of the front and back sections of the outer cover, and attached to the other one of the front and back sections of the outer cover. In other examples, the front or back section of the outer cover may be provided with one single or with a pair of side portions being attachable so as to form a closed circumference, i.e. a belt, around the wearer, whereafter the opposite front or back section of the outer cover may be attached to the one or more side portions.

The attachment between the side portions or between an side portion and a front or back section of the outer cover may be made via waist attachment means. For example, the side portions may be provided with waist attachment means for attachment to corresponding waist attachment means provided on another side portion, or on the front or back section of the outer cover, for closing the article around the waist of a wearer.

For example, when a pair of side portions is arranged in one out of the front and back sections of the outer cover, the inward facing side of the side portions may be provided with waist attachment means for attachment to corresponding attachment means provided on the outward facing side of the opposite front or back section, respectively, of the outer cover, for closing the article around the waist of a wearer.

In certain examples, the back section of the outer cover may be provided with a pair of side portions, each side portion extending transversally on each side of a main body, and intended to be fastened to the front section of the outer cover. For example, the inward facing side of the side portions may be provided with waist attachment means for attachment to corresponding attachment means provided on the outward facing side of the front section of the outer cover, for closing the article around the waist of a wearer.

### Waist Attachment Means of the Outer Cover

Generally, in the front or back section of the outer cover, the inward facing surface of the garment portion may be provided with a pair of waist attachment means to be fastened to a corresponding waist attachment means on the outward facing surface of the garment portion in the opposite back or front section, respectively. For example, in the back section of the outer cover, the inward facing surface of the garment portion may be provided with a pair of waist attachment means to be fastened to a corresponding waist attachment means on the outward facing surface of the garment portion.

The waist attachment means may comprise matching attachment areas, for example fastener areas or attachment areas similar to those described in the below in relation to the insert fastener areas of the outer cover, or to the cover fastener areas of the insert. For example, the waist attachment means may comprise corresponding hooks and loops.

### Properties of the Outer Cover

The outer cover is reusable, i.e. intended to be used for more than one use cycle of an absorbent article. As such, at the start of each use cycle, a new absorbent insert may be attached to the outer cover to form the absorbent article, and after each use cycle, the absorbent insert may be removed from the outer cover and disposed. The outer cover may be configured to be cleanable between use cycles. As such, the outer cover may if necessary be cleaned between each use cycle. However, it may be preferred that the outer cover is only cleaned when deemed necessary, i.e. when, after one or more use cycles, the outer cover is deemed to be soiled so as to require cleaning.

To provide for efficient cleaning, the outer cover may be configured to be washable.

For example, the outer cover may be configured to be machine-washable.

For example, the outer cover may be configured for at least 20 wash cycles., such as for at least 20 machine-wash cycles as defined herein.

Optionally, the outer cover may be configured for at least 50 wash cycles, such as for at least 50 machine-wash cycles as defined herein.

Optionally, the outer cover may be configured for at least 20 machine-wash cycles, such as at least 50 machine-wash cycles at 60 degrees Celsius.

Washing allows for hygienic cleaning of the outer cover, further increasing the reusability of said cover. Being machine washable is preferred, as this makes the product easier for caregivers to clean and reuse.

As mentioned in the above, the outer cover is not primarily intended to contain bodily discharges such as urine or faeces from the wearer. Instead, the absorbent insert, which is to be attached to the outer cover when the absorbent article is in use, is to perform the function of absorbing bodily discharges from the wearer.

As such, the outer cover may be mainly liquid permeable.

The outer cover being mainly liquid permeable implies that the outer cover is substantially permeable to liquids, such as urine.

As such, at least 50% of a total area of the outer cover may be liquid permeable. For example, at least 70% or at least 90 % of a total area of the outer cover may be liquid permeable.

That the outer cover may be mainly liquid permeable implies that suitable materials for the outer cover may be selected among liquid permeable materials.

Thus, the outer cover may be made by liquid permeable materials, such as textile fabrics. This provides for the absorbent article resulting by the combination of the outer cover and an absorbent insert having a more underwear-like look and feeling.

The outer cover may be mainly breathable. The outer cover being breathable, i.e. gas permeable, implies that air and other gases may pass through the outer cover. This is beneficial for the skin health of the wearer.

As such, at least 50% of a total area of the outer cover may be breathable. For example, at least 70% or at least 90 % of a total area of the outer cover may be breathable.

Preferably, the outer cover may be mainly liquid permeable and mainly breathable. As such, the outer cover may be made by liquid permeable, breathable materials such as for example textile materials.

Suitably, the outer cover may be mainly liquid permeable, mainly breathable, and washable as set out in the above. To that end, suitable materials being liquid permeable, breathable, and washable may be selected to form the outer cover. Again, suitable materials may be found among textile materials being liquid permeable, breathable, and washable.

For example, the garment portion of the outer cover may comprise one or more fabric panels forming the front section, the back section, and the crotch section of the garment portion.

As such, the one or more fabric panels may be selected from materials being liquid permeable, breathable, and/or washable.

For example, the one or more fabric panels may comprise a textile fabric. Suitable textile fabrics may be liquid permeable, breathable and/or washable.

For example, the one or more fabric panels may comprise a knitted textile fabric.

A knitted textile fabric may be suitable to provide elasticity to the outer cover, as discussed in the below.

For example, the one or more fabric panels may comprise a weft knit textile fabric material, for example as a weft knit single jersey.

For example, the outer cover may comprise a textile fabric, having a basis weight in the range between 150 and 300 gsm, such as between 200 and 250 gsm. For example, the one or more fabric panels may comprise a textile fabric having a basis weight in the range between 150 and 300 gsm, such as between 200 and 250 gsm. Such a fabric could be a knitted textile fabric, such as a weft knit textile fabric, e.g. a weft knit single jersey as set out in the above.

The outer cover may comprise a material that is elastic in at least one direction, preferably in two directions. For example, the one or more fabric panels may comprise a material that is elastic in at least one direction, preferably in two directions. Such a material could be e.g. a knitted fabric as set out in the above.

For example, the outer cover may comprise a textile fabric comprising polyester.

As such, the one or more of the fabric panels of the garment portion may comprise a textile fabric comprising polyester. For example, the majority of the area of the garment portion may comprise a textile fabric comprising polyester, such as at least 60% of the area, or at least 80 % or at least 90 % or the area.

For example, the one or more fabric panels may comprise a textile fabric comprising a mixture of polyester and elastane. For example, one or more fabric panels may comprise a textile fabric comprising no less than 70% polyester, such as no less than 80% polyester. For example, one or more fabric panels may comprise a textile fabric comprising 70 to 90% polyester, and 30 to 10% elastane. For example, the one or more fabric panels may comprise a fabric comprising 80% polyester and 20 % elastane.

Polyester materials have been found to have advantageous properties throughout use and washing cycles, for example in relation to shrinking, retaining its shape and stretch.

For example, the outer cover may comprise a textile fabric comprising recycled material. As such, one or more of the fabric panel or panels of the garment portion may comprise a textile fabric comprising recycled material. For example, the majority of the area of the garment portion may comprise a textile fabric comprising recycled material, such as at least 60% of the area, or at least 80 % or at least 90% or the area.

For example, the amount of recycled material in the textile fabric may be at least 20%, such as at least 50%, or at least 70%.

In an example, the garment portion may comprise one single fabric panel forming the front section, the back section and the crotch section of the garment portion.

In another example, the garment portion comprises more than one fabric panel, the fabric panels being joined to form the front section, the back section, and the crotch section of the garment portion. Use of more than one fabric panel may provide for further options for controlling the shape of the outer cover and/or other properties, such as elasticity, of the outer cover.

The fabric panels may be joined by any suitable joining members. For example, the joining members could be selected considering the requirements for e.g. washability and/or breathability of the outer cover.

For example, the fabric panels may be joined by adhesive joining members, such as adhesive tape.

For example, the fabric panels may be joined by mechanical joining members such as stiches or seams.

The fabric panels may be permanently joined, i.e. intended to be joined throughout the life cycle of the outer cover.

Optionally, when the garment portion comprises more than one fabric panel, the fabric panels may be made by different fabrics. As such, different properties of different fabrics may be used to impart different functions and/or aesthetics to the garment portion and to the outer cover.

In another option, when the garment portion comprises more than one fabric panel, the fabric panels may be made by the same fabric. As such, the fabric panels will have similar properties, i.e. liquid permeability and/or breathability, and similar behaviour when subject to use, such as washing and wearing. This may be advantageous for the durability of the reusable outer cover. Moreover, using the same fabric material for the fabric panels may provide for efficient use of fabric material during manufacture, reducing fabric material waste.

As mentioned in the above, the absorbency of the absorbent article to be formed by the outer cover, when an absorbent insert is attached thereto, is primarily to be provided by the absorbent insert alone.

For example, the crotch portion of the outer cover may be essentially non-absorbing, implying that the crotch portion is not intended to absorb any bodily discharges to a significant degree.

For example, the garment portion of the outer cover may be essentially non-absorbing, and/or the entire outer cover may be essentially non-absorbing.

With essentially non-absorbing is meant herein that the outer cover is not intended to absorb and retain fluids to a significant degree.

To this end, the one or more fabric panels of the garment portion and/or of the outer cover may consist of one or more materials, each material being essentially non-absorbing.

### Insert Fastener Areas of the Outer Cover

As set out in the above, in accordance with the second aspect of the disclosure, in at least one, preferably each, of the front section and the back section of the outer cover, the outer cover comprises at least one flap member extending from the garment portion. Each flap member has a use position wherein an outward facing inner side of the flap member is facing the inward facing side of the garment portion so as to form a space between the outward facing inner side of the flap member and the inward facing side of the garment portion, which space is open in a longitudinal direction towards the crotch section for receiving a respective back edge portion and/or a front edge portion of the insert therein. Further, the outer cover comprises a set of one or more insert fastener areas being positioned on the outward facing inner side of the at least flap member. Each insert fastener area in the set comprises a surface fastening material for detachably attaching an insert to the outer cover.

Thus, in the front section and/or back section of the garment portion, a flap member is arranged such that, at least in a use position, the flap member delimits a space being open in a longitudinal direction towards the crotch section for receiving a respective back edge or a front edge of the insert. The space is open in a longitudinal direction towards the crotch section. As such, a back edge or a front edge of an absorbent insert may be positioned in the space, and the inward facing side of the insert may be attached by the one or more insert fastener areas (each insert fastener area comprising a surface fastening material), while the absorbent insert extends out of the space and over the crotch section of the outer cover, so as to be available for absorbing bodily fluids when the absorbent article is in use.

The one or more insert fastener areas in said set are positioned on the outward facing side of the at least flap member, facing towards the garment portion. A second side of the flap member, opposite the outward facing inner surface, is hence, in the use position, an inward facing surface facing towards the intended wearer.

The flap member may be a generally planar member, extending in the plane spanned by the longitudinal axis and the transversal axis.

As such, when the outer cover is in use, the one or more insert fastener areas positioned on the outward facing side of the flap member will be directed away from the wearer, in a direction towards the garment portion of the outer cover. Accordingly, any risk of the wearer experiencing chafing or discomfort from the surface fastening material of the insert fastener areas is significantly reduced-even if the insert should accidentally loosen from the insert fastener areas. Also, that the insert fastener areas are positioned on the outward facing side of the flap member implies that the flap member will be held securely towards the inward facing side of insert when the article is in use. As such, any risk of the flap member becoming unintentionally folded or creased, creating possible discomfort for the wearer, is reduced.

Moreover, with the one or more insert fastener areas being positioned on the flap member, any undesired aesthetical effects on the outer cover, resulting from the insert fastener area(s), (such as for example seams attaching an insert fastener member to outer cover), will appear on the flap member only and not be visible from the outside of the garment portion, when the outer cover is worn. The outward facing surface of the garment portion is intended to be the outward facing surface of the complete absorbent article, and hence it will be directed towards a wearer's regular garments when the outer cover is in use.

Further, that the one or more insert fastener areas is/are positioned on the outward facing surface of the flap member, which surface is spaced from the garment portion, implies that, when the outer cover is in use, the movement of the garment portion is less restricted by the insert fastener area and thus the insert. For example, any area of the garment portion over which the flap member extends will be able to follow the general movement of the garment portion, and so, the outward appearance of the garment portion will be more underwear-like, without visible tension in the garment portion resulting from the attachment of the insert to the outer cover.

Similarly, the insert fastener areas, and thus the position of the insert, will be less prone to being dislocated by e.g. the garment portion being subject to tearing or friction, resulting from the wearer's movements.

By the definition in the above, the set of one or more insert fastener areas comprises all insert fastener areas in the front or back section, respectively, which are positioned on the outward facing inner side of the at least one flap member and comprises a surface fastening material for detachably attaching an insert.

Possibly, the outer cover could comprise additional fastener areas for providing detachable attachment between the outer cover and the insert, in addition to the set of insert fastener areas positioned on the outward facing inner side of the at least one flap member. For example, an additional fastener area could be provided on the garment portion of the outer cover. However, any such additional fastener areas should preferably be minimised, since the additional attachment may impair the benefits obtained by using the set of one or more insert fastener areas positioned on the outward facing inner side of the flap as proposed herein.

Rather, in the at least one, preferably each, of the front section and the back section of the outer cover, the set of one or more insert fastener areas positioned on the outward facing inner side of the at least one flap member may be the only insert fastener elements for detachably attaching an insert to the outer cover in said front and/or back section of the outer cover.

Preferably, each of the front section and the back section of the outer cover comprises a set of insert fastener areas positioned on the outward facing inner side of the at least one flap member, wherein said sets of insert fastener areas are the only insert fastener elements for detachably attaching an insert to the outer cover, of the outer cover.

The outward facing side of the flap member may face at least a portion of the inward facing surface of the garment portion, i.e. face the area of the wearer facing inward facing surface over which the flap member extends. Thus, the space may be formed between the outward facing side of the flap member and the inward facing surface of the garment portion.

For example, the area of the inward facing surface of the garment portion over which the flap member extends (when in the use position) may be free from insert fastener areas. Optionally, the entire garment portion may be free from insert fastener areas. As such, the only attachment between the outer cover and an insert may be made via the flap members comprising the sets of insert fastener areas, which may be configured for fastening towards the inward facing side of the insert.

### Extension of Insert Fastener Areas

In said at least one, preferably each, of the front section and the back section, the set of insert fastener areas may span over a maximum longitudinal length being no more than 20% of the longitudinal length of the outer cover, such as no more than 15% of the longitudinal length of the outer cover, or no more than 10% of the longitudinal length of the outer cover.

Optionally, the set of one or more insert fastener areas may be confined to a front or back end region of the outer cover, said front or back end region extending longitudinally from a front or back edge of the outer cover and over a maximum longitudinal length being no more than 20% of the longitudinal length of the outer cover, such as no more than 15% of the longitudinal length of the outer cover, or no more than 10% of the longitudinal length of the outer cover.

As such, the attachment of the insert to the outer cover is confined to the front or back end regions of the front or back section of the outer cover. Accordingly, a front or back edge region may be held securely by the outer cover when the article is worn, while a crotch section of the insert may be unattached to the outer cover.

### Flap Member of the Outer Cover

For example, in said at least one, preferably each, of the front section and the back section, each flap member may be located at a front edge or a back edge, respectively, of the outer cover.

As such, a flap member in the front section may extend longitudinally from a front edge of the outer cover towards the crotch section. Similarly, a flap member in the back section may extend longitudinally from a back edge of the outer cover towards the crotch section.

Thus, the insert may be attached at a location in the front and/or back section of the outer cover close to the front edge and/or back edge of the outer cover, respectively. As such, the position of the insert fastener areas, and hence of the insert when attached to the outer cover, may be secured.

As such, in said at least one, preferably each, of the front section and the back section, each flap member may for example have a longitudinal length being no more than 20% of the longitudinal length of the outer cover, such as no more than 15% of the longitudinal length of the outer cover, or no more than 10% of the longitudinal length of the outer cover.

With the longitudinal length of the flap member is meant the maximum longitudinal length of the flap member as extending freely in the longitudinal direction, when in the use position. For example, the free length may be measured in a longitudinal direction between a joint attaching the flap member to the garment portion, and a free end of the flap member.

With the longitudinal length of the outer cover is meant the maximum longitudinal length from the front edge to the back edge of the outer cover, as measured via the crotch section of the outer cover. In other words, the longitudinal length is the maximum longitudinal length as seen over the crotch width of the outer cover. As such, portions of the outer cover extending transversally beyond the crotch width may be disregarded when determining the longitudinal length of the outer cover. For example, transversally extending side portions provided to provide fastening of the outer cover around the waist of the wearer may be disregarded. The longitudinal length of the outer cover is to be measured when any flap members of the outer cover is/are in their use position.

Optionally, each of the front section and the back section comprises at least one flap member, and a sum of the maximum longitudinal lengths of the flap members in the front section and the back section is no more than 30% of the total longitudinal length of the outer cover, such as no more than 20% of the total longitudinal length of the outer cover.

With the sum of the maximum longitudinal lengths is meant the sum of the maximum longitudinal length of a flap member in the front section, and the maximum longitudinal length of a flap member in the back section.

The flap member being relatively short as compared to the longitudinal length of the outer cover, implies that, when in the use position, the flap member will leave a substantial section of the inward facing side of the outer cover uncovered, as seen from the inward direction of the outer cover. Hence, in use, the insert attached to the outer cover will form the inward facing surface of the absorbent article over this substantial section. Thus, the flap members will leave a substantial section of the inward facing surface of the insert free to receive bodily discharge from the wearer. Further, in the crotch portion of the absorbent article, the inward facing surface of the outer cover (the crotch portion of the outer cover), will be substantially covered by the insert, which protects the outer cover from being soiled.

For example, each flap member may have a longitudinal length that is less than a longitudinal length of the respective front or back section in which it is arranged. Thus, each flap member may be confined to the respective front and back section, leaving the crotch section of the outer cover free.

The outer cover may be such that, when each flap member is in the use position, the garment portion is not covered by any flap member (as seen from the inward facing side of the outer garment) over at least 50% of the longitudinal length of the outer cover. For example, the garment portion is not covered by any flap member over at least 70%, or over at least 80%, of the longitudinal length of the outer cover,

As such, over at least 50% of the longitudinal length of the outer cover, such as over more than 70%, or over at least 80%, of the longitudinal length of the outer cover, the wearer facing side of the garment portion forms the wearer facing side of the outer cover when each flap member of the outer cover is in a use position.

The longitudinal length of the flap member may be selected so as to provide sufficient area for the insert fastener area on the garment facing inward facing surface of the flap member, while still not covering more of the wearer facing side of the insert than necessary.

In said at least one, preferably each, of the front section and the back section, each flap member may have an area being no more than 20% of the area of the outer cover, such as no more than 15% of the area of the outer cover. Preferably, each flap member may have an area being no more than 10% of the area of the outer cover.

A total area of all the flap members of the outer cover may be no more than 40 % of the area of the outer cover. For example, the total area of the flap members may be no more than 30% of the total area of the garment portion. Preferably, the total area of the flap members may be no more than 20% of the total area of the garment portion.

At least one, such as each, of the one or more insert fastener areas may each have an area smaller than the area of the respective flap member at which it is provided. In addition, the sum of the areas of all insert fastener areas on a flap member may be smaller than the area of the flap member.

The advantages of the limited areas of the flap members/insert fastener areas as set out in the above in relation to the flap member, the outer cover, or the garment portion, are similar to those explained in the above in relation to the lengths.

### Joining of Flap Member to Garment Portion

As set out in the above, in the front and/or back section of the outer cover, at least one flap member extends from the garment portion.

The at least one flap member may extend from the garment portion via one or more joints and/or one or more folds. In other words, the at least one flap member may be connected to the garment portion via one or more joints and/or one or more folds.

For example, each flap member may be formed by a separate piece of material, such as a fabric panel, being connected to the garment portion via one or more joints, such as seams, welds, adhesive joints or the like. Preferably, the one or more joints may be seams.

In another example, the flap member may be formed by a portion of a piece of material which is also forming a part of the garment portion. For example, a fabric panel may be folded such that on one side of the fold, it forms part of the garment portion, and on the other side of the fold, it forms the flap member.

Also, in some examples, a flap member may extend from the garment portion via one or more folds and one or more joints.

The at least one flap member has a use position wherein an outward facing inner side of the flap member is facing the inward facing side of the garment portion so as to form a space between the outward facing inner side of the flap member and the inward facing side of the garment portion, which space is open in a longitudinal direction towards the crotch section.

To this end, when in a use position, the flap member may comprise a free edge being directed towards the crotch section, which free edge is unattached to the garment portion, so as to allow introduction of a back portion or a front portion of an insert into the space.

The flap member extends from the free edge, in a direction longitudinally towards the front or back end of the outer cover, to a front or back edge, respectively.

For example, the flap member may extend from the garment portion at least along the back or front edge, respectively of the flap member. In other words, the flap member may be connected to the garment portion at least along the back or front edge. As mentioned in the above, the connection may be made via e.g. a fold or one or more joints.

Further, the flap member may comprise transversally spaced side edges, connecting said free edge and said front/back edge of the flap member. The side edges may extend in a generally longitudinal direction along the outer cover.

For example, the flap member may extend from the garment portion at least along the transversally spaced side edges of the flap member. In other words, the flap member may be connected to the garment portion at least along said transversally spaced side edges. As mentioned in the above, the connection may be made via e.g. one or more folds and/or one or more joints. The free edge of the flap member being directed towards the crotch section of the outer cover may extend between the transversally space side edges of the flap member.

As explained in the above, each flap member has a use position wherein the outward facing inner side of the flap member is facing a space being open in a longitudinal direction towards the crotch section for receiving a respective back edge and/or a front edge of the insert therein.

In some variants, a flap member may have a non-use position, wherein the flap member does not face or delimit such a space. For example, in a non-use position, the flap member may be folded such that side of the flap member which is the outward facing side in the use position, i.e. the side of the flap member which carries the insert fastener areas, is facing towards the inward facing direction. For example, the flap member could be in such a non-use position while an absorbent insert is arranged with its outward facing side towards the inward facing side of the outer cover. Once the insert is correctly positioned in relation to the outer cover, the flap member could be brought to the use position, forming the space receiving the edge of the insert.

For example, a flap member having a non-use position could be a flap member extending from the garment portion only along a back or front edge, respectively, of the flap member, and not along the transversally spaced side edges.

However, optionally, the flap member is permanently in the use position.

For example, through the flap member extending from the garment portion at the transversally spaced side edges, the use position of the flap member may be a default position wherein the flap member is held down towards the garment portion. In use, the front and/or back edge of the insert may be introduced into the space of the flap member between first and second side joints or folds provided at the transversally spaced side edges.

Thus, the set of insert fastener areas on the outward facing side of the flap member is covered, preventing any accidental contact between the surface fastening material of the insert fastener areas and the wearer's skin.

Moreover, by the flap member being connected to the garment portion at the side edges, it may be ensured that the flap member is held in position when the outer cover is worn.

Further, for each flap member, it may be desired that the flap member extends from the garment portion at at least a pair of positions, wherein one position of the pair is located on a first transversal side of a longitudinal centre axis of the garment portion and the other position of the pair is located on a second transversal side of the longitudinal centre axis of the garment portion. (The longitudinal centre axis of the garment portion is a longitudinal centre axis through the crotch portion of said outer cover.)

When the outer cover is worn, the front and back sections thereof will be subject to opposing transversal forces in directions away from a longitudinal centre axis of the outer cover, due to the attachment of the outer cover around the waist of the wearer. Via the flap member being connected to at least one position on each side of the central longitudinal axis, the opposing transversal forces applied to the garment portion may be transferred to the flap member, ensuring that the flap member is stretched in the transversal direction and held close to the body of the wearer.

For example, when, as mentioned in the above, the flap member extends from the garment portion at least along a front/back edge, respectively, of the flap member, the fold or joint forming the connection to the garment portion may extend transversally over the longitudinal centre axis of the outer cover, such that the flap member is connected to at least one position on each side of the longitudinal axis.

For example, when, as mentioned in the above, the flap member extends from the garment portion at least at the two transversally spaced side edges, one side edge may be positioned on one transversal side of the longitudinal centre axis of the outer cover, and the other side edge may be positioned on the other transversal side of the longitudinal centre axis of the outer cover.

As mentioned in the above, in the front and/or back section of the outer cover, the main body may be provided with one or more transversally extending side portions, for providing attachment of the outer cover around the waist of a wearer. For example, the flap member may be arranged to extend from the garment portion such that the transversal forces applied to the transversally extending side portions are transmitted via to the flap member.

When the flap member extends from the garment portion at least at the two transversally spaced side edges, the flap member may be connected to the garment portion along a longitudinal length of each side edge at least being such that the connection spans over the length spanned by the set of insert fastener areas of the flap member. For example, the connection may span over essentially the full longitudinal length of the two transversally spaced side edges.

In an example, the flap member may extend from the garment portion at a back/front edge and at the transversally spaced side edges, such that the space between the inward facing surface of the flap member and the wearer facing side of the garment portion is open in a longitudinal direction towards the crotch section only. As such, the correct positioning of the back edge or front edge of the insert, respectively, in the space will be facilitated. Also, the advantages as set out in the above relating to the permanent use position and to the distribution of forces may be obtained.

The material of the flap member may be selected so as to be washable, to the same extent as the material of the garment portion of the outer cover, and as explained in the above. Further, although the flap members will cover a relatively small area of the wearer when the article is in use than the garment portion, it may still be desired that the flap members are breathable and/or liquid permeable.

As such, the material for the flap member may be selected to be a material being washable, breathable and/or liquid permeable. As such, the material properties and materials discussed in the above in relation to the outer cover as a whole or to the garment portion are preferable also for the flap members.

For example, each of the flap member(s) may comprise one or more fabric panels, in the same manner as described in the above in relation to the garment portion of the outer cover. For example, the flap member(s) may each comprise one single fabric panel. This may be preferred for ease of manufacture.

Preferably, the one or more insert fastener areas may be entirely contained within the one or more fabric panels of the flap member, preventing any direct contact between the fastener areas and the wearer's skin. For example, the flap member(s) may comprise one or more fabric panels and one or more insert fastener areas being contained within the one or more fabric panels. By “entirely contained” is meant that the one or more insert fastener aeras do not extend beyond, e.g. does not protrude from, the one or more fabric panels.

In other examples, the flap member(s) may comprise one or more fabric panels and one or more insert fastener areas, wherein optionally the one or more insert fastener areas extend beyond the one or more fabric panels.

For example, the insert fastener areas may be provided by insert fastener members attached to the one or more fabric panels, as will be further described in the below.

Similar to the garment portion, the flap member may comprise a textile fabric, preferably a knitted fabric. As such, the flap member may comprise one or more fabric panels having any of the features as set out in the above in relation to the one or more fabric panels of the garment portion.

Advantageously, the garment portion and the flap member may comprise one or more fabric panels, wherein at least some of the fabric panels of the garment portion is of the same material as the fabric panel of the flap member. For example, the garment portion and the flap member may comprise one or more fabric panels, wherein all fabric panels are of the same material.

As outlined in the above, in said at least one, preferably each, of the front and back section of the outer cover, at least one flap member is provided. Accordingly, in some variants, a plurality, such as two or more flap members may be arranged in said at least one, preferably each, of the front and back section of the outer cover. In this case, the one or more insert fastener areas of the set of insert fastener areas may be distributed over the plurality of flap members, such that each of the two or more flap members comprises one or more insert fastener areas, positioned on the respective outward facing sides of the flap members. For example, two or more transversally spaced flap members could be arranged in the front section and/or in the back section of the outer cover.

However, it may be preferred that a single flap member is arranged in the front section of the outer cover, and/or a single flap member is arranged in the back section of the outer cover.

In this case, all of the one or more insert fastener areas of the set will be positioned on the outward facing side of the single flap member.

The single flap member may for example have a transversal width being no less than a minimum transversal width defined by the crotch section of the outer cover. For example, the single flap member may have a transversal width being at least 120% of the minimum transversal width of the outer cover of the outer cover. Optionally, the single flap member may have a transversal width being at least 130% of the minimum crotch width of the outer cover, such as at least 140% of the minimum crotch width of the outer cover.

As such, the single flap member may have a transversal width adapted to be greater than the transversal width of an insert to be used with the outer cover.

For example, when the garment portion comprises a main body and transversally extending side portions in the front and/or back section, the single front and/or back flap member may extend over the width of the main body. For example, the single front and/or back flap member may be attached to the garment portion by side joints, where the side joints may for example be formed together with joints attaching the transversally extending side portions to a main body of the garment member.

For example, a single flap member may be joined to the garment portion at a front/back joint, and a pair of side joints. As such, the entire front/back edge of an insert may be introduced in the space formed by the single flap member for attachment of the insert to the outer cover. Thus, simple and correct positioning of the insert may be enabled. Moreover, the single flap member being joined to the garment portion at the front/back joint and the pair of side joints ensures that there are no unnecessary free edges of the flap member, which could be prone to cause chafing.

For example, the front section and the back section of the outer cover may each comprise a set of insert areas and a single flap member comprising the set of inert areas, wherein the total area of the single flap member in the front section and the single flap member in the back section is no more than 40% of the area of the outer cover, such as no more than 30% or no more than 20%.

The single flap member may comprise the set of one or more insert fastener areas, as will be described in more detail in the below.

For example, the single flap member may comprise a first and a second subset of insert fastener areas, the first and the seconds subset being transversally spaced.

### Transversal Fastening Width of the Outer Cover

In said at least one, preferably each, of the front section and the back section of the absorbent article, the set of one or more insert fastener areas spans over a transversal fastening width.

The crotch section of the outer cover defines a minimum transversal width of the outer cover. Optionally, in the at least one, preferably each of the front section and the back section of the outer cover, the set of one or more insert fastener areas spans over a respective transversal fastening width being larger than the minimum transversal width.

As such, the set of one or more insert fastener areas allows attachment of the insert in a position such that the insert is transversely held out to a sufficient width in the front and/or back section, respectively, while in at least part of the crotch section, the insert may adapt to a cup shape generally within the minimum transversal width of the outer cover.

For example, the respective transversal fastening width may be no less than 110% of the minimum transversal width, such as no less than 120% of the minimum transversal width (crotch width) of the outer cover.

The outer cover may comprise a pair of leg elastic members, arranged along the leg openings of the garment portion at least in the crotch section thereof.

The leg elastic members may be separated by a maximum leg elastics spacing width. The maximum leg elastics spacing width is the maximum transversal width between the elastically active portions of the leg elastic members.

Similarly, the leg elastic members are separated by a minimum leg elastics spacing width. As such, the minimum leg elastics spacing width is typically located in the crotch section of the garment portion, and the minimum leg elastics spacing width may be close to the minimum transversal width (crotch width) of the outer cover.

For example, in said at least one, preferably each of, the front section and the back section of the outer cover, the set of one or more insert fastener areas spans over a respective transversal fastening width being less than the maximum leg elastics spacing width. As such, a desired positioning of the leg elastic members of the outer cover transversally outside of an insert attached to the set of one or more insert fastener areas may be ensured.

For example, when, in said at least one, preferably each of, the front section and the back section of the outer cover, the set of one or more insert fastener areas spans over a respective transversal fastening width being less than the maximum leg elastics spacing width and larger than the minimum transversal width of the outer cover, correct positioning of the insert in relation to the outer cover when the absorbent article is in use may be promoted.

Thus, as may be understood from the above, the crotch section of the outer cover may advantageously be free from insert fastener areas. Indeed, the outer cover as a whole may be free from insert fastener areas, but for said set of one or more insert fastener areas in at least one, preferably each, of the front section and the back section of the outer cover.

### Subsets of Insert Fastener Areas

In said at least one, preferably each, of the front section and the back section of the outer cover the set of one or more insert fastener areas may comprise a first subset of one or more insert fastener areas and a second subset of one or more insert fastener areas, wherein the first subset and the second subset are spaced around a longitudinal central axis of the outer cover by a transversal spacing width. Preferably, the first and second subset may be symmetrically spaced around the longitudinal axis by the transversal spacing width.

As such, the insert may be free from fastener areas over the transversal spacing width. This implies that the insert may be free from attachment to the outer cover in a central transversal region, which may be beneficial for forming the insert during use. For example, the spaced arrangement may allow the insert to assume a slight cup-shape when seen in the transverse direction, improving the inserts retention capacity. Further, the spaced arrangement of the insert fastener areas allow for arrangements where correct positioning of the insert to the outer cover may be facilitated. Moreover, when the insert is free from insert fastener areas over the transversal spacing width, the surface fastening material of the insert fastener areas is provided only where needed, implying that the amount of material on the outer cover may be reduced.

As mentioned in the above, the crotch section of the outer cover may define a minimum transversal width (the crotch width). For example, the transversal spacing width may no less than the minimum transversal width. For example, the transversal spacing width may be at least 120% of the minimum transversal width.

As such, the first and second subsets of one or more insert fastener areas may ensure that the insert, when attached to the fastener areas, is extending at least over the crotch width of the outer cover.

For example, the transversal spacing width may be at least three times the transversal width of each insert fastener area, or at least four times the transversal width of each insert fastener area.

For example, the transversal spacing width may be no less than 40% of the transversal fastening width, such as no less than 50% or no less than 60% of the transversal fastening width.

For example, in said at least one, preferably each, out of the front section and the back section, the first and second subsets of one or more insert fastener areas may be provided on the same flap member, preferably on a single flap member in the front or back section, respectively.

For example, the front section and the back section of the outer cover may each provided with a single flap member, each single flap member comprising a first subset of one or more insert fastener areas and a second subset of one or more insert fastener areas, the first and second subsets being spaced, preferably symmetrically spaced, around a longitudinal central axis of the insert by a transversal spacing width. For example, the first subset and the second subset may each comprise one single fastener area.

In one example, the subsets of insert fastener areas may be provided by one single insert fastener member, attached to the single flap member. For example, such an insert fastener member may comprise a sheet formed base carrying a first subset of one or more insert fastener areas comprising a surface fastening material and a second subset of one or more insert fastener areas comprising a surface fastening material, the first and second subsets being spaced by the transversal spacing width.

However, in another example, each one of the insert fastener areas may be provided by a separate insert fastener member, each insert fastener member comprising one insert fastener area comprising a surface fastening material. This may be advantageous to reduce the amount of material added to the insert, and/or to reduce any additional stiffness implied by the presence of an insert fastener member on the outer cover.

Further features and advantages of the outer cover will be apparent from the above description of an absorbent article, and the below description of an insert for forming an absorbent article. It is to be understood that the features relating to an outer cover, or to the insert fastener areas of an outer cover as described in relation to the absorbent article in the above, are applicable also to the outer cover per se, alone or in combination.

### 3.Absorbent Insert

In a third aspect, the disclosure relates to an absorbent insert according to claim 57.

Thus, the disclosure relates to an absorbent insert for use with an outer cover for forming an absorbent article adapted to be worn about a wearer's waist. The insert is shaped to, in use, cover a wearer's pubic area and a predefined part of the buttocks. The insert extends along a longitudinal direction, and along a transversal direction, perpendicular to the longitudinal direction, wherein an inward facing direction is defined being towards the wearer when the absorbent article is worn, and an outward facing direction is defined being away from the wearer when the article is worn, the inward facing and outward facing directions being perpendicular to the transversal and longitudinal directions.

The insert has a longitudinal length that extends along a longitudinal direction and the insert is subdivided into a front section, a back section and a crotch section along said longitudinal length. The insert comprises a liquid impermeable backsheet, a liquid permeable topsheet, and an absorbent assembly provided in between said backsheet and said topsheet.

The insert has an inward facing side which comprises the topsheet to be directed towards the wearer, and an outward facing side to be directed towards the outer cover, when the article is in use. In at least one, preferably each, of the front section and the back section of the insert, the insert comprises a set of one or more cover fastener areas being provided on the inward facing side of the insert. Each cover fastener area comprises a surface fastening material for detachably attaching said insert to said outer cover.

Thus, it is proposed herein to provide an absorbent insert which, in at least one, preferably each, of the front section and back section, comprises a set of one or more cover fastener areas on the inward facing side of the insert, thus enabling detachable connection to an outer cover to form an absorbent article via the inward facing side of the insert, as outlined in the above.

The one or more cover fastener areas may be different from any other areas on the inward facing side of the insert, i.e. the surface fastening material of each of the one or more cover fastener areas may be different from any other materials on the inward facing side of the insert. For example, the surface fastening material of the one or more cover fastener areas may be different from the topsheet material and/or a leakage barrier forming material on the inward facing side of the insert. As such, the connection between the outer cover and the insert may be controlled as regards for example the location of the connection and/or the strength of the connection formed.

As such, e.g. the position and/or the surface area and/or the type of surface fastening material of the one or more cover fastener areas may be adapted to the insert fastener areas of the outer cover, so as to provide a suitable detachable connection of the insert to the outer cover. For example, as mentioned in the description of the absorbent article in the above, the insert fastener areas of the outer cover may be adapted so as to provide for a greater attachment force when attached to the cover fastener areas, than to any other areas of the inward facing side of the insert. For example, as mentioned in the description of the absorbent article in the above, the insert fastener areas of the outer cover may be adapted so as to be non-adherend to any areas of the inward facing side of the insert but the cover fastener areas. This may facilitate handling and ensure that the insert is properly positioned in relation to the outer cover.

The one or more cover fastener areas may be arranged so as not to protrude beyond the extension of the backsheet of the insert. That is to say, that the complete one or more cover fastener areas may be arranged so as to overlap the remainder of the insert, rather than on any extension, flap or the like protruding from the insert per se.

Further features relating to the cover fastener areas are described in the above in relation to the attachment areas.

The insert could optionally be configured to be washable.

However, the insert may preferably be configured for single-use. In other words, the insert may preferably be disposable.

### General Construction of Absorbent Insert

### Topsheet

As stated in the above, the insert comprises a liquid impermeable backsheet, a liquid permeable topsheet, and an absorbent assembly provided between the topsheet and the back sheet.

Materials suitable for topsheets are commonly known in the art of disposable absorbent hygiene products, and for the purposes of the present disclosure any material commonly known for use as a topsheet material may be used, including, but not limited to non-woven materials and perforated polymeric films.

The topsheet is suitably sufficiently liquid permeable to allow discharged body fluids such as urine to penetrate through the thickness of the topsheet. Also, the topsheet is suitably manufactured from a material which is compliant and soft feeling to the skin of the wearer.

The topsheet may be manufactured from various web materials such as woven and nonwoven webs, perforated films, open cell foams, or combinations or laminates of the above-mentioned materials.

A nonwoven material suitable as a topsheet can be manufactured from synthetic fibres such as polyester or polypropylene, or natural fibres such as cotton fibres. A mix of synthetic and natural fibres may also be used.

The nonwoven materials to be used for the topsheet may for example be made of a spunbond, a spunbond/spunbond composite or a spunbond/meltblown composite, such as a SMS, SSMS, SSMMS, SMMS, nonwoven material of polypropylene or bicomponent fibers of polypropylene and polyethylene, or of a combination of such materials. The topsheet may also have elastic properties.

The topsheet may be hydrophilized in order to improve the tendency for urine to penetrate the topsheet into the underlying structures. Methods for hydrophilizing nonwovens are known and include coating the nonwoven material with a hydrophilic coating, such as by applying a surfactant coating; by applying a hydrophilic monomer composition and a radical polymerization initiator onto the nonwoven followed by initiating a polymerization reaction on the nonwoven; by applying a coating of hydrophilic nanoparticles; or by treating the nonwoven surface with a high energy treatment.

A surfactant coating may be obtained for example by applying a surfactant composition to the non-woven material by any suitable means including spraying, slot coating, kiss roll coating and/or soaking the material in a bath containing the surfactant. The hydrophilization treatment may be performed in-line during assembly of the absorbent hygiene product, or may performed separately and the topsheet may then delivered as ready-to-use rolls to the disposable absorbent hygiene product manufacturing plant.

The topsheet material may have a basis weight of from 8 to 20 g/m2 , such as from 12 to 17 g/m2 . However, the disclosure is not limited to topsheet materials having this basis weight only.

### Backsheet

Materials suitable as backsheets are commonly known in the art of disposable absorbent hygiene products. The backsheet may be substantially impermeable to liquids, such as urine.

The backsheet may be breathable. A breathable, i.e. gas permeable, back sheet is beneficial for the comfort and skin health of the wearer. As the insert disclosed herein is intended for use with an outer cover as disclosed herein to form an absorbent assembly, use of a breathable backsheet may enable an absorbent assembly to be formed, which assembly exhibits satisfactory breathability.

The backsheet may be substantially liquid impermeable and breathable, i.e. gas permeable, implying that air and other gases may pass through the backsheet while being substantially impermeable to liquids.

For the purposes of the present disclosure, any material commonly known for use as a backsheet material may be included in the backsheet, including but not limited to polymeric films, for example films of polyethylene, polypropylene or copolymers of polyethylene or polypropylene, hydrophobized nonwoven materials, fluid impermeable foams and fluid impermeable laminates. The backsheet may comprise one or more layers of material. For example, the backsheet may be a laminate of a liquid impermeably polymeric film towards the absorbent assembly and nonwoven towards the garment side, to provide a textile, soft feeling to the outward facing surface of the disposable absorbent hygiene product.

It is also contemplated that the backsheet may be made or otherwise include an entirely or partially elastic material in order to give the product a better fit when in use.

However, with the present insert, it is believed to be advantageous if the backsheet is a comprises a nonwoven, for example that the backsheet is a laminate of a porous polymeric film and a nonwoven.

### Absorbent Core

The absorbent assembly can comprise one or more absorbent cores. The cores can be constructed from one or more layers of cellulose fluff pulp. The cellulose fluff pulp can be mixed with fibres or particles of a highly absorbent polymer material, so-called superabsorbent polymers, of the type that chemically binds large quantities of fluid on absorption with the formation of a fluid-holding gel. The absorbent core can also comprise highly absorbent polymer material arranged in a layer inside the absorbent core or connected to the surface or surfaces of the absorbent core. The absorbent core can further include further components for improving the properties of the absorbent core. Examples of such components are binding fibres, various types of fluid-dispersing layers or fibres, dimensionally stabilising components, reinforcing fibres or the like.

Superabsorbent polymers are well-known in the field of absorbent products and are used to help improve the absorbent properties of such products. Superabsorbent polymers are constituted by water-swellable and water-insoluble polymers that are capable of absorbing large quantities of fluid upon formation of a hydrogel, such as capable of absorbing at least 5 times their weight of an aqueous 0.9 % saline solution as measured according to the method NSWP 241.0.R2. The superabsorbent polymer polymers for use in accordance with the present disclosure may be inorganic or organic crosslinked hydrophilic polymers, such as polyvinyl alcohols, polyethylene oxides, crosslinked starches, guar gum, xanthan gum, crosslinked polyacrylates, and the like. The polymers may be in the form of powders, granules, microparticles, films, foams and fibers, for example. Upon contact with fluids, such super absorbent polymers swell by absorbing the fluids into their structures. In general, super absorbent polymers can quickly absorb fluids insulted into such products, and can retain such fluids to prevent leakage and help provide a dry feel even after fluid insult.

The type of super absorbent polymer used in an absorbent assembly of the embodiments discussed herein may be the same or may vary within the assembly. For example, a super absorbent polymer with a first set of characteristics may be used in the front and back regions of the absorbent assembly, or in a first absorbent core, and a super absorbent polymer with a second set of characteristics may be used in the central region of the absorbent assembly, or in a second absorbent core. The characteristics referred to in this section is for example the centrifuge retention capacity, absorption under load and/or the gel layer permeability.

Thus, the absorbent assembly of the insert as proposed herein may advantageously comprise super absorbent polymers. For example, the absorbent assembly may comprise super absorbent polymers and cellulosic fibers.

For example, the absorbent assembly may comprise a super absorbent polymer concentration of at least 15 weight %, based on the total weight of cellulosic fibers and super absorbent polymer.

The absorbent assembly may comprise a first absorbent layer and a second absorbent layer, the first and second absorbent layers being stacked in between the backsheet and the topsheet.

As such, super absorbent polymers may be present in one or both of the first and second absorbent layers.

### Aquisition Layer

Further, the insert may include an acquisition layer applied between the topsheet and the absorbent assembly. Materials suitable as acquisition layers, also referred to in the art as transfer layer, or ADL, are commonly known in the art of disposable absorbent hygiene products, and for the purposes of the present disclosure, any material known to the person skilled in the art as being useful as an acquisition layer may be used. An acquisition layer may for example be in the form of an airlaid layer, a spunlace layer, a high-loft, foam or any other type of material layer which may be used in an absorbent hygiene product to act as a liquid acquisition and absorption layer. The acquisition layer is suitably adapted to quickly receive and temporarily store discharged liquid before it is absorbed by the absorbent core. Such acquisition layer may be composed of for example airlaid nonwoven, spunlace nonwoven, high loft nonwoven or foam materials. An airlaid nonwoven may be produced with fluff, wood pulp, and here the fluff fibres are dispersed into a fast-moving air stream and condensed onto a moving screen by means of pressure and vacuum. The acquisition layer may preferably be of an air-through bonded nonwoven of polyester fibers.

### Further Properties of the Insert Relating to the Absorbent Assembly

The absorbent assembly may be mainly positioned in the crotch section of the insert and have an absorbent assembly length that spans no more than 70% of the longitudinal length of the insert, such as no more than 85% of the longitudinal length of the insert.

Thus the absorbent assembly does not extend all the way to the front and back edge of the insert, preventing liquid from being stored close to the edges which are positioned inside the flap members of the outer cover. This reduces the risk of soiling of the outer cover, allowing the outer cover being reused without the need of being cleaned.

For example, the detachable cover fastener areas may be confined to the portions of the insert extending longitudinally beyond the absorbent core towards the front edge or back edge, respectively. As such, the attachment of the insert to the outer cover is constant, regardless of whether the insert has absorbed any amount of liquid or not.

The absorbent core may be hour-glass shaped, having a narrowest width in the transverse direction in a longitudinal central position of the crotch section of the insert.

With hour-glass shaped is meant herein that the absorbent core has a narrowest width in the crotch section of the insert, and that a width of the absorbent core towards the front section and towards the back section, respectively, is greater than the narrowest width.

In other alternatives, the absorbent core may have other shapes, such as a rectangular or oval shape.

Optionally, the longitudinal edges of the insert are partially corresponding to the hour-glass shape of the absorbent assembly.

As such, the insert may have a shape with a narrowest width in the transverse direction in the crotch section of the insert.

In other alternatives, the insert may have a rectangular shape.

The insert may have a length along the longitudinal axis being greater than a width along the transversal axis.

For example, the insert may have total absorption capacity of at least 1000 ml, such as from 1000 to 4000 ml. As such, the insert is an insert suitable for use as in an absorbent article being an incontinence article suitable for adult wearers.

### Cover Fastener Areas

As set out in the above, in the third aspect of the invention there is provided an absorbent insert and the insert having an inward facing side comprising said topsheet to be directed towards the wearer, and an outward facing side to be directed towards the outer cover, when the article is in use, wherein at least one, preferably each, of the front section and the back section of the insert comprises a set of one or more cover fastener areas being provided on the inward facing side of the insert, each cover fastener area comprising a surface fastening material for detachably attaching said insert to said outer cover.

As such, when the insert is attached to an outer cover, the insert may be held securely in place allowing for the benefits regarding for example comfort and aesthetic effects as outlined in the above in relation to the outer cover. For example, when the insert is attached to an outer cover and worn by a wearer, the cover fastener areas will be covered by the remainder of the insert, and so they will be protected from being torn or plucked from the outside of the absorbent article.

Similarly, the cover fastener areas, and thus the position of the insert, will be less prone to being dislocated by e.g. tearing or friction, resulting from the wearer's movements.

By the definition in the above, the set of one or more cover fastener areas comprises all cover fastener areas in the front or back section, respectively, which are positioned on the inward facing side of the insert and comprises a surface fastening material for detachably attaching an outer cover.

Possibly, the insert could comprise additional fastener areas for providing detachable attachment between an outer cover and the insert, in addition to the set of cover fastener areas positioned on the inward facing side of the at least insert. For example, an additional fastener area could be provided on the outward facing side of the insert. However, any such additional fastener areas should preferably be minimised, since the additional attachment may impair the benefits obtained by using the set of one or more cover fastener areas positioned on the inward facing side of the insert as proposed herein.

Rather, in the at least one, preferably each, of the front section and the back section of the insert, the set of one or more cover fastener areas positioned on the inward facing side of the insert may constitute the only fastener elements in said front and/or back section of the insert being adapted for detachably attaching the insert to an outer cover.

Preferably, each of the front section and the back section of the insert comprises a set of cover fastener areas positioned on the inward facing side of the insert, wherein said sets of cover fastener areas constitute the only fastener elements of the insert being adapted for detachably attaching the insert to an outer cover.

Optionally, the insert extends longitudinally between a front edge and a back edge, and in said at least one, preferably each, out of the front section and the back section of the insert, the one or more cover fastener areas in the set are positioned at or adjacent to the respective front and back edge of the insert.

Through being located near the front and back edges, the one or more cover fastener areas are easily covered completely by the flap members of the outer cover as described in the above, preventing any contact with the wearer's skin during use.

When the one or more cover fastener areas in the set are positioned at the respective front or back edge of the insert, advantages in manufacturing may be obtained, for example when the insert comprises one or more cover fastener members which may be cut in line with the front or back edge of the insert in manufacture.

Optionally, in said at least one, preferably each, of the front section and the back section (of the insert, the set of one or more cover fastener areas spans a longitudinal length, which is no more than 20% of the longitudinal length of the insert, such as no more than 10% of the longitudinal length of the insert.

As such, when the insert in use is attached to an outer cover via a set of one or more cover fastener areas in one or each of the front section and the back section, the majority of the longitudinal length of the inward facing side of the insert will be available for absorbing bodily fluids, while the set of one or more cover fastener areas provides for secure attachment of the insert to the outer cover.

Further, in said at least one, preferably each, of the front section and the back section of the insert, a front or back edge portion, respectively, may be defined as the portion of the insert extending from the respective front or back edge and over the longitudinal length spanned by said set of one or more cover fastener areas. As such, when the insert is attached to an outer cover as described in the above, a flap member provided with a set of one or more insert fastener areas will suitably be adapted to extend at least partially over said front or back edge portion of the insert.

Optionally, in said at least one, preferably each, of the front section and the back section of the insert, the front/back edge portion may have an edge portion length being less than 20% of the longitudinal length of the insert, such as less than 10% of the longitudinal length of the insert.

When the set of one or more cover fastener areas extends from the front or back edge, respectively, the edge portion length is thus equal to the longitudinal length spanned by the one or more cover fastener areas.

As mentioned in the above, the absorbent assembly may be mainly positioned in the crotch section of the insert and have an absorbent assembly length. For example, the absorbent assembly length may span no more than 70% of the longitudinal length of the insert, such as no more than 85% of the longitudinal length of the insert.

Further, in said at least one, preferably each, out of the front section and the back section of the insert, the front or back end, respectively, of the absorbent assembly may be spaced from the front or back edge portion, respectively, by an absorbent spacing length (Lac). The absorbent spacing length may for example be at least 3% of the longitudinal length of the insert, such as at least 4% or at least 5% of the longitudinal length of the insert. For example, the absorbent spacing length may be from 3% to 10% of the longitudinal length of the insert, such as from 5% to 10% of the longitudinal length of the insert.

Thus, it may be ensured that the swelling of the absorbent assembly when taking up fluids will not interfere with the connection of the insert to the outer cover.

Optionally, a sum of a longitudinal length spanned by any one or more cover fastener areas in the front section and a longitudinal length spanned by any one of more cover fastener areas in the back section of the insert is equal to or less than 30% of the longitudinal length of the insert, such as equal to or less than 20% of the longitudinal length of the insert.

As such, when the insert in use is attached to an outer cover via a set of one or more cover fastener areas in each of the front section and the back section, the majority of the longitudinal length of the inward facing side of the insert will be available for absorbing bodily fluids, while the set of one or more cover fastener areas provides for secure attachment of the insert to the outer cover.

Similarly, a sum of the edge portion length of the front edge portion and the edge portion length of the back edge portion may be equal to or less than 30% of the longitudinal length of the insert, such as equal to or less than 20% of the longitudinal length of the insert.

Optionally, in said at least one, preferably each, of the front section and the back section of the insert, each of the one or more cover fastener areas in said set has an area being no more than 10% of the area of the insert, such as no more than 5% of the area of the insert, or no more than 2% of the area of the insert.

As such, when the insert in use is attached to an outer cover via a set of one or more cover fastener areas in each of the front section and the back section, the majority of the total area of the inward facing side of the insert will be available for absorbing bodily fluids, while the sets of one or more cover fastener areas provides for secure attachment of the insert to the outer cover.

Optionally, a total area of all of the one or more cover fastener areas of the insert is no more than 25% of the area of the insert, such as no more than 10% of the area of the insert.

As such, when the insert in use is attached to an outer cover via a set of one or more cover fastener areas in each of the front section and the back section, the majority of the longitudinal area of the inward facing side of the insert will be available for absorbing bodily fluids, while the set of one or more cover fastener areas provides for secure attachment of the insert to the outer cover.

### Transversal Fastening Width of the Insert

In the at least one, preferably each, of the front and back section, the set of one or more cover fastener areas spans a transversal fastening width.

In some variants, when a set of one or more cover fastener areas is present in each of the front and back sections, the transversal fastening width of the set of one or more cover fastener areas in the front section may be the same as the transversal fastening width of the set of one or more cover fastener areas in the back section.

In some variants, when a set of one or more cover fastener areas is present in each of the front and back sections, the transversal fastening width of the set of one or more cover fastener areas in the front section may be different from the transversal fastening width of the set of one or more cover fastener areas in the back section.

Optionally, in the at least one, preferably each, of the front and back section, the transversal fastening width spanned by the set of one or more cover fastener areas is at least 70% of the maximum width (Wi) of the insert in said front or back section, respectively, such as at least 80% or at least 90% of the maximum width. Thus, when the insert is attached to an outer cover via the set of one or more cover fastener areas, it may be ensured that insert is stretched out over at least the majority of the maximum width of the insert in the front and/or back section, respectively. As such, correct positioning of the insert in relation to a wearer may be ensured.

Thus, when the insert is attached to an outer cover via the set of one or more cover fastener areas, it may be ensured that insert is stretched out over a width of the insert in the front and/or back section, respectively, which is sufficient to ensure that a large enough width of the inner surface of the insert is facing towards the wearer when the article is in use. As such, correct positioning of the insert in relation to a wearer may be ensured.

The transversal fastening width may be no less than the minimum transversal width in the crotch section of the insert, such as no less than 110% of the minimum transversal width or no less than 120% of the minimum transversal width.

### Insert Leg Elastic Members

The insert extends longitudinally between a front edge and a back edge, and comprises longitudinal side edges connecting the front edge and the back edge. Further, the insert may comprise a pair of insert leg elastic members extending essentially along the longitudinal direction of the insert, and arranged on both sides of the insert, in between the absorbent assembly and the longitudinal side edges of the insert. For each of the front and back section of the insert, a leg elastics width is defined as the maximum transversal width over the insert leg elastic members in said front or back section, respectively. Optionally, the leg elastics width in the front and back section of the insert may be the same, and correspond to a maximum transversal width over the insert leg elastic members.

The insert leg elastic members contribute to forming the insert around the user's body, and to provide sufficient fluid tightness at the leg openings of the absorbent article when attached to a wearer. Further, the insert leg elastic members contribute to providing correct positioning of the insert when the insert is in use. As such, the use of insert leg elastic members may contribute to providing sufficient fluid tightness when the insert is in use as attached to an outer cover, such that the outer cover may be liquid impermeable.

In said at least one, preferably each, out of the back section and the front section, the transversal fastening width spanned by the one or more cover fastener areas in the set may be no less than the leg elastics width in said back section or front section, respectively.

The transversal fastening width being no less than the leg elastics width implies that the insert leg elastics may be stretched out in a transversal direction when the insert is attached to the outer cover, thus ensuring proper function and positioning of the insert leg elastics.

Optionally, in said at least one, preferably each, out of the back section and the front section of the insert, the insert may comprise a pair of leg edge portions, each leg edge portion extending longitudinally along a respective insert leg elastic member, and transversally between the insert leg elastic member and a longitudinal side edge of the insert so as to define a maximum transversal leg edge width between said insert leg elastic member and said respective longitudinal side edge, the maximum transversal leg edge width being at least 1 cm, such as at least 2 cm.

For example, the maximum transversal leg edge width may be no more than 4 cm.

Herein, a maximum transversal leg edge width is defined in each of the front section and the back section of the absorbent article. Optionally, the maximum transversal leg edge width may be the same in the front section and in the back section of the absorbent article, and may correspond to a maximum transversal leg edge width of the insert.

The leg edge portions extending transversally between the insert leg elastic members and the longitudinal side edge of the insert provides for additional leakage security of the absorbent article. In use, the leg edge portions are to extend transversally outwards from the insert leg openings as formed at the insert leg elastic members.

When combined with the transversal fastening width spanned by the set of cover fastener areas being no less than the leg elastics width, as set out in the above, the leg edge portions may be at least partially extended in a transversal direction when the insert is attached to the outer cover. As such, the leg edge portions may be automatically folded out to a correct position when the insert is attached to the outer cover. Thus, the set of cover fastener areas contributes to folding out and holding the leg edge portion in a transversally extending state during fastening of the absorbent article to a wearer, and during use thereof. Accordingly, manual manoeuvres otherwise required for folding out the leg edge portion may be dispensed with. This is a benefit in particular for adult wearers, improving the dignity of the wearer in the care situation by avoiding manual manipulation of leg edge portions located at the crotch region of the wearer.

For example, the transversal fastening width may span into at least 50% of the transversal width of each leg portion, such as at least 75% of the transversal width of each leg portion, or at least 80% of the transversal width of each leg portion.

Optionally, the insert leg elastic members are sandwiched between an innermost layer of the insert and the backsheet.

The insert leg elastic members extend along a longitudinal insert leg elastics length. The insert leg elastics length may be at least 50% of the total longitudinal length of the insert, such as along at least 75% or at least 85% of the total longitudinal length.

The insert leg elastics length is to be defined as the longitudinal length of the insert along which the leg elastic members are active. I.e. any portions where the leg elastic members are fastened or deadened such that they no longer provide elasticity to the insert is to be disregarded.

(Similarly, the insert leg elastics width as defined in the above is to be measured over the parts of the leg elastic members which are active.)

For example, the insert leg elastics length may extend along at least 60 % of a total longitudinal length of the absorbent core, such as along at least 80%, or at least 90 % of the total longitudinal length of the absorbent core.

Optionally, in said at least one, preferably each, of the front section and the back section of the insert, the set of one or more cover fastener areas is confined to a longitudinal section of the insert not spanned by the leg elastics length.

As such, the function of the cover fastener areas to ensure extension of the insert leg elastic members in the longitudinal direction may be improved. For example, the set of one or more cover fastener areas may be confined to a longitudinal section of the insert not spanned by the leg elastics length.

As such, when the transversal fastening width is greater than the leg elastics width, and each cover fastener area is positioned longitudinally in a section between the front/back end of the insert leg elastic members and the front/back edge of the insert, the cover fastener areas may, when the insert is in use and attached to an outer cover, ensure stretching in transversal and longitudinal directions of the insert leg elastic members.

Each insert leg elastic member may comprise one or more elastic elements. For example, the elastic elements may comprise one or more elastic threads, elastic film strips or elastic foam strips. The elastic elements may be attached to the insert when in a stretched state so as to provide elasticity to the insert.

With the longitudinal extension of the insert leg elastic members, i.e. the insert leg elastics length is meant herein any extension of the leg elastic members which is active, i.e. along which elasticity is indeed provided by the insert leg elastic member. Accordingly, any portions of elastic elements wherein the elastic is disabled are to be disregarded when determining the extension of the leg elastics.

### Inner Side Leakage Barriers of the Insert

The inward facing side of the insert may comprise a pair of inner side leakage barriers, extending in the longitudinal direction of the insert.

Inner side leakage barriers are also often referred to as “standing gathers”.

The side leakage barriers, may extend generally along, and at a distance from the respective longitudinal side edges of the insert.

The inner side leakage barriers may be adapted to be moveable between a first position, in which the inner side leakage barriers form a flat surface that is parallel to the topsheet, and a second position, in which the inner side leakage barriers are raised up from the liquid-permeable topsheet, over at least 60% of the longitudinal length of the insert, spaced from both a front edge and a back edge of the insert.

A side leakage barrier length may be defined as the longitudinal length over which the side leakage barriers are free to rise over the liquid-permeable top sheet. As mentioned in the above, the side leakage barrier length may be at least 60% of the longitudinal length of the insert. The side leakage barrier length may for example extend over the full extension of the absorbent assembly.

However, so as not to interfere with the connection of the insert to the outer cover, the side leakage barrier length may be arranged so as not extend into the back and/or front edge portion of the insert. For example, the side leakage barrier length may be spaced from the back and/or front edge portion of the insert. For example, the side leakage barrier length may be spaced from the back and/or front edge portion of the insert by at least 5% of the total insert length, such as by at least 7% of the total insert length.

For example, the side leakage barrier length may be less than 80% or the longitudinal length of the insert, such as less than 70%.

Each inner side leakage barrier may comprise an elastic element. For example, the elastic element may be one or more elastic threads.

Each side leakage barrier may comprise a sheet material and an elastic element, such as an elastic thread. For example, each side leakage barrier may comprise more than one elastic thread, such as 2 to 5 elastic threads. The sheet material of each side leakage barrier may comprise two layers of material. The sheet material of each side leakage barrier may be attached to the top sheet. The attachment may be a longitudinal welding line and/or a line of adhesive. The sheet material of each side barrier may comprise a part being attached to the topsheet and a part being non-attached to the topsheet comprising the elastic thread. The elastic thread of the first and second side leakage barrier respectively may be raised above the inward facing surface of the topsheet. The elastic thread of each standing gather may be raised for example at least 20 mm above the inward facing surface of the topsheet, such as from 20 mm to 70 mm, or from 30 mm to 50 mm.

The sheet material may be a hydrophobic material. For example, the sheet material may be a hydrophobic nonwoven material, such as a nonwoven material being coated with a hydrophobic coating. Thus, each side leakage barrier may be a hydrophobic side leakage barrier.

Preferably, the insert comprises insert leg elastic members as described in the above and inner side leakage barriers as described in the above. As such, the pair of inner side leakage barriers will be arranged transversally closer to the longitudinal axis Y than the pair of insert leg elastic members.

A maximum width between each inner side leakage barrier in the pair of inner side leakage barriers is defined herein as a barrier width. The barrier width is the maximum transversal width as measured between the locations of each side leakage barrier at which the raiseable portions of the leakage barrier is attached to the remainder of the inward facing side of the insert.

Thus, the barrier width may for example be less than the leg elastics width.

The width of the raisable part of each side barrier (the part being non-attached to the topsheet) may be less than half the barrier width—i.e. the side barriers are not intended to completely cover the topsheet between them, when in a folded down state. Instead, the width of the raisable part of each side barrier may be e.g. less than 30% of the barrier width, such as less than 25% of the barrier width. For example, the width of the raisable part of each side barrier may be from 15 to 25% of the barrier width.

An insert comprising a pair of inner side leakage barriers as exemplified in the above and a pair of insert leg elastic members as exemplified in the above will hence provide double barriers against leakage of bodily fluids from the insert when in use. As such, the insert is particularly useful together with an outer cover as described herein. For example, due to the insert being capable of ensuring reliable acquisition and absorption of the bodily fluids, the outer cover may be made liquid permeable, as the function of the outer cover is to provide for correct positioning of the insert to the wearer, rather than to provide for fluid permeability and/or absorption of fluids. As such, the outer cover may be made by liquid permeable materials, such as textile fabrics. This provides for the absorbent assembly formed by the outer cover with an insert attached thereto having a more underwear-like look and feeling. Moreover, increased use of liquid permeable, breathable materials such as textile materials towards the body of the wearer provides for improved skin health.

### Subsets of Cover Fastener Areas

Optionally in said at least one, preferably each, out of the back section (Sb) and the front section (Sf), said set of one or more cover fastener areas comprises a first subset of one or more cover fastener areas and a second subset of one or more cover fastener areas, wherein the first and second subsets are spaced around the longitudinal central axis of the insert by a transversal spacing width. Preferably, the first and second subsets may be symmetrically spaced around the longitudinal central axis by the transversal spacing width.

Accordingly, the insert may be free from cover fastener areas over a spacing width as measured transversely between said subsets of a areas.

This contributes to the shaping of the insert when in use, as attached to an outer cover forming an absorbent article applied to a wearer. Notably, the front and/or back section of the insert may be held out over the transversal fastening width, so as to secure the position of the insert visavi the wearer, while allowing for relative movement between the outer cover and the insert over the spacing width. This allows for the insert to shape in relation to the body of the wearer, for example, to assume a slight cup-shape. Further, the spaced arrangement of the cover fastener areas allow for arrangements where correct positioning of the insert to the outer cover may be facilitated. Moreover, when the insert is free from insert fastener areas over the transversal spacing width, the surface fastening material of the insert fastener areas is provided only where needed, implying that the amount of material on the outer cover may be reduced.

As explained in the above the set of cover fastener areas may span over a transversal fastening with being close to the maximum width of the insert in the front or back region, respectively, such as over a transversal fastening with being at least 80% of the maximum width of the insert in the front or back region, respectively. Thus, when combined with the set of cover fastener areas comprising a first subset and a second subset being spaced by the spacing width, it is understood that the first and second subset may each be positioned adjacent a respective longitudinal side edge of the insert.

For example, the transversal spacing width may be at least 50% of the maximum width of the insert at the respective back or front section, such as at least 70% of the maximum width or at least 80% of the maximum width.

The transversal fastening width is naturally to be greater than the transversal spacing width. For example, the transversal fastening width may greater than the transversal spacing width by a distance corresponding to at least 10% of the maximum width of the insert at the respective back or front section.

For example, the transversal spacing width may be at least three times the transversal width of each cover fastener area, or at least four times the transversal width of each cover fastener area.

For example, the transversal spacing width may be at least 40% of the transversal fastening width, such as at least 50% or at least 60% of the transversal fastening width. Thus, the location of the connection between the insert and the outer cover may be controlled such that the function of the insert in use is ensured, while at the same time enabling use of cover fastener aeras having sufficient size to provide a sufficiently strong connection.

For example, when the absorbent insert comprises a pair of inner side leakage barriers being spaced apart as mentioned in the above by a barrier width, the transversal spacing width may be at least said barrier width.

For example, when the absorbent insert comprises a pair of inner side leakage barriers are spaced apart, symmetrically around the longitudinal axis, by a barrier width, and a pair of insert leg elastic members extending over a maximum leg elastics width, the set of one or more cover fastener areas may span over a transversal fastening width such that the leg elastics width is greater than the barrier width, and the transversal fastening width is equal to or greater than the maximum leg elastics width. Optionally, when the absorbent insert further comprises a first and second subset of one or more cover fastener areas, symmetrically spaced around the longitudinal axis by a spacing width, the spacing width may for example be greater than or equal to the barrier width.

The proposed positioning of the cover fastener areas implies as set out in the above that correct tensioning of the insert leg elastic members is assured when the insert is attached to the outer cover. Further, the proposed positioning also implies that correct tensioning and thus raising up of the side leakage barriers is assured when the insert is attached to the outer cover. Thus, the proposed positioning of the cover fastener areas improves the leak-proof performance of the absorbent article and facilitates the process of positioning the absorbent article on a wearer.

As explained in the above, the insert is preferably to be attached to the outer cover in a state in which it is stretched out flat as seen in a transversal direction, and in at least one, preferably each, out of the front edge portion and the back edge portion of the insert.

Thus, at least over the respective transversal fastening width, the front edge portion and/or the back edge portion may suitable be free from folds. For example, the entire the front edge portion and/or the back edge portion may suitable be free from folds.

Accordingly, the front edge portion and/or the back edge portion of the insert comprising for example the topsheet, leakage barrier forming material, and the backsheet, is free from folds. As such, the front edge portion and/or the back edge portion is adapted to be attached to the outer cover via the set of one or more cover fastener members in an unfolded condition.

For example, when the respective set of cover fastener members comprises two subsets of cover fastener members spaced by a transversal spacing width, the front edge portion and/or the back edge portion may be free from elastic members providing elasticity in a transversal direction of the insert over said transversal spacing width.

For example, at least over the respective transversal fastening width, the front edge portion and/or the back edge portion may be free from elastic members providing elasticity in a transversal direction of the insert over said transversal spacing width. For example, the entire front edge portion and/or the back edge portion may be free from elastic members providing elasticity in a transversal direction of the insert.

For example, the insert as a whole may be free from elastic members providing elasticity in a transversal direction of the insert.

By elastic members providing elasticity in a transversal direction of the insert is meant elastic members, such as elastic threads or the like, added to the remainder of the insert e.g. to the assembly of the topsheet, leakage barrier forming material, backsheet, or absorbent assembly, so as to, when activated retract the remainder of the insert in a transversal direction.

### Subsets of Cover Fastener Areas in Relation to Cover Members

For example, in said at least one, preferably each, out of the front section and the back section, the first and second subsets of one or more cover fastener areas may be provided by one single cover fastener member, the cover fastener member thus having a surface comprising a surface fastening material forming the two subsets of one or more cover fastener areas.

However, preferably, the first subset of one or more cover fastener areas may be provided by a first subset of one or more cover fastener members, and the second subset of one or more cover fastener areas may be provided by a second subset of cover fastener members. Accordingly, the first and second subset of cover fastener members may be transversally spaced by the spacing width. This is advantageous to reduce the amount of material added to the insert. Further, the correct positioning of the insert visavi the outer cover may be better controlled, by the one or more cover fastener areas providing fewer options for fastening to the outer cover by this arrangement.

For example, the insert may comprise one single first cover fastener member providing the first subset of one or more cover fastener areas, and one single second cover fastener member providing the second subset of one or more cover fastener areas.

For example, each one out of the first and second subsets may comprise only one single cover fastener area.

For example, the insert may comprise one single first cover fastener member providing one single cover fastener area, and one single second cover fastener member providing one single cover fastener area.

Further features and advantages of the insert will be apparent from the above description of an absorbent article, and the above description of an outer cover for forming an absorbent article. It is to be understood that the features relating to an inert, or to the cover fastener areas of an inert as described in relation to the absorbent article in the above, are applicable also to the insert per se, alone or in combination.

### 4.Packaging

In a fourth aspect, there is provided a packaging in accordance with claim 92. As such, there is provided a packaging comprising a plurality of inserts according to the third aspect as disclosed herein.

The packaging may further comprise an outer cover according to the second aspect as disclosed herein.

The present disclosure is to encompass all of the aspects as described herein, alone or in combination.

As such, the disclosure is to encompass for example an absorbent article according to the first aspect, wherein the outer cover is in accordance with the second aspect and/or the insert is in accordance with the third aspect as described herein, as combined with any of the applicable features.

Several variants are encompassed by the various aspects of the invention, as understood by the above description.

Moreover, it will be understood that when referring to a feature that exists in each of the front section or the back section of the article, the feature in the front section need not be identical to the feature in the back section. For example, a width of a member in the front section need not be identical to a width of a member in the back section, even if both widths are to fulfil the same general requirement.

In the following, variants of the different aspects (absorbent article, outer cover, insert, and packaging) of the invention will be described with reference to exemplary embodiments and to the drawings. It is to be understood that the drawings are schematical and that the items depicted are not necessarily drawn to scale.

### BRIEF DESCRIPTION OF THE DRAWINGS

Variants of an absorbent article, outer cover, and insert will be described by way of example, with reference to the attached drawings, in which:

FIG. 1 shows an embodiment of an absorbent article according to the invention;

FIG. 2a shows an embodiment of an insert according to the invention;

FIG. 2 b shows a section through the horizontal axis X of the insert in FIG. 2a;

FIG. 2c shows a section through the line c-c of the insert in FIG. 2a;

FIG. 3a shows the outer cover of the absorbent article depicted in FIG. 1;

FIG. 3b shows the outer cover of FIG. 3a with the flap members shown as removed from the remainder of the outer cover;

FIGS. 4a to 4d depict the use-cycle of an absorbent article similar to that depicted in FIG. 1, respectively showing a package containing at least an insert, the attachment of an insert into an outer cover, the absorbent article being worn by a person and the removal and disposal of an insert and cleaning of the outer cover.

In the drawings, for reasons of simplicity, reference numbers of some features (e.g. widths or lengths) are noted for only one out of a front and back section of an absorbent article, an outer cover, or an insert, respectively. However, it is to be noted that similar measures may be applied to the other one out of the front and back sections. It is further to be noted that even if a feature is denoted with the same reference number in the front section and in the back section, dimensions such as widths and lengths of such features may differ between the front and the back section.

### DESCRIPTION OF VARIANTS

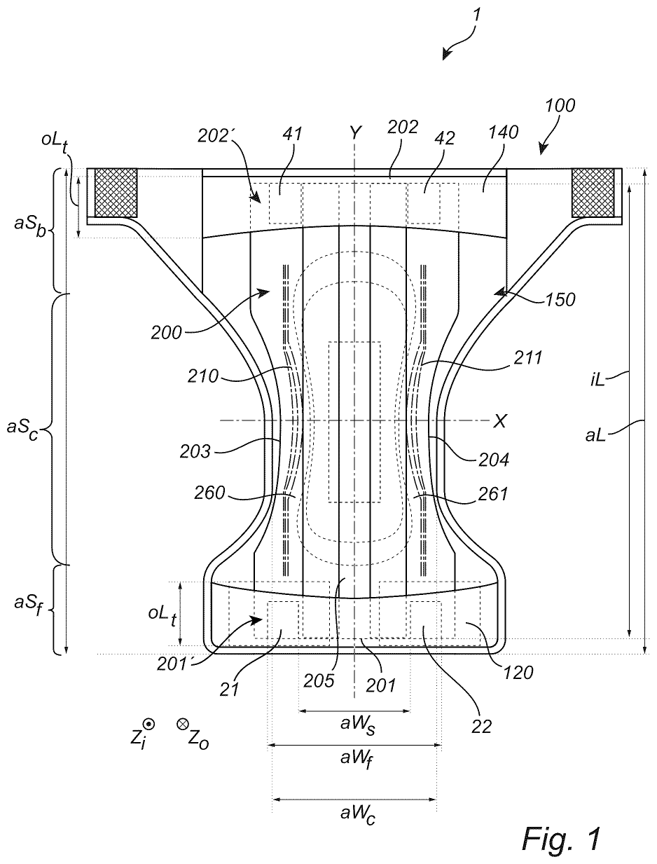

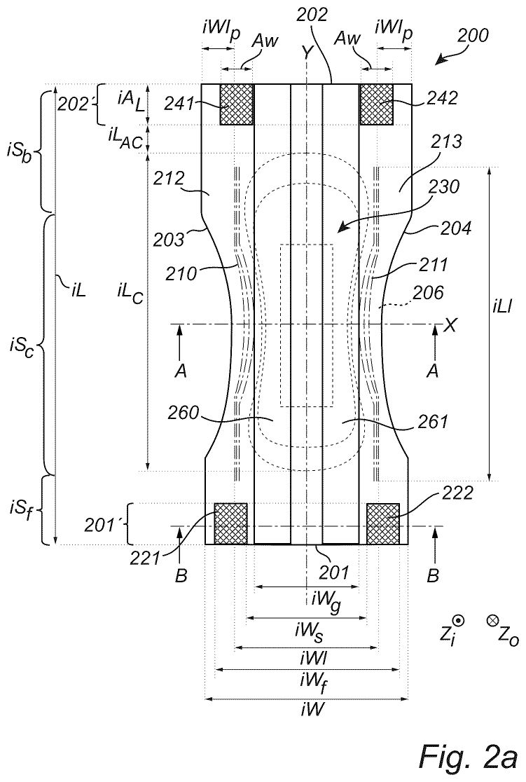

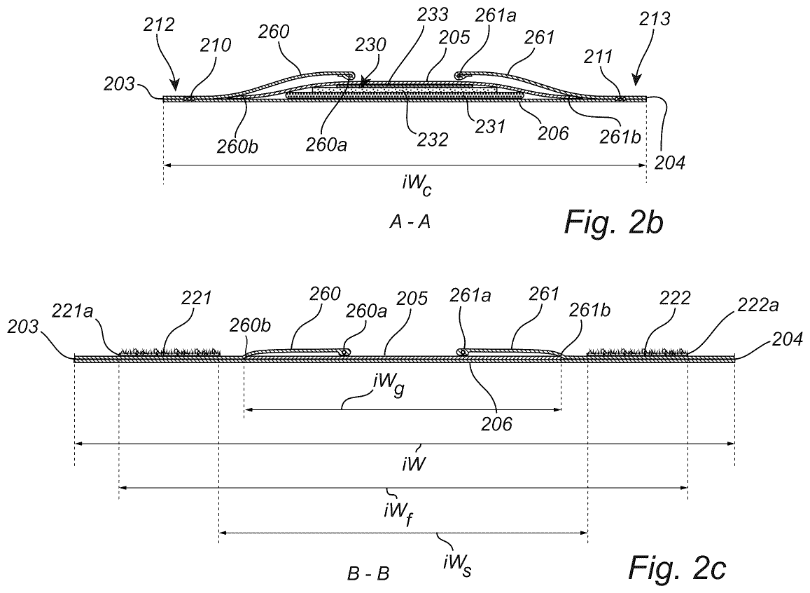

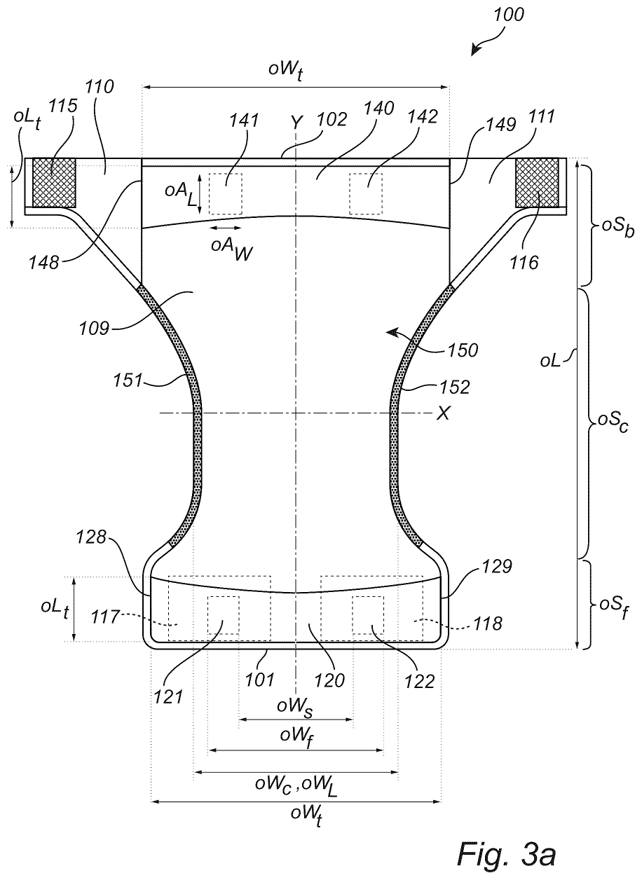

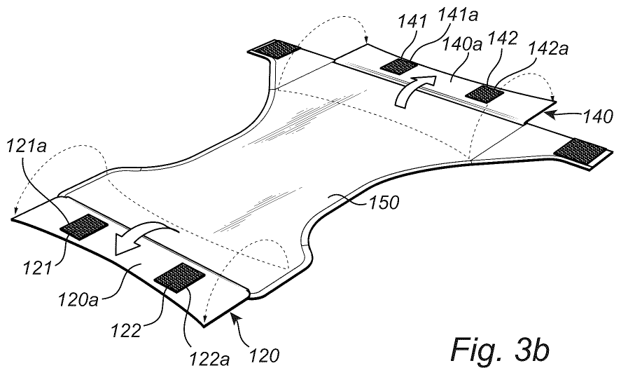

Absorbent articles as defined by the appended claims consist of a reusable outer cover and an insert. An embodiment of such an absorbent article 1 is shown in FIG. 1, having reusable outer cover 100 and insert 200, which are respectively shown independently from one another in more detail in FIGS. 3a, 3b and 2. The absorbent article 1 exhibits a longitudinal direction X and transverse direction Y and has a thickness direction Z, which, when the article is worn, defines an inward direction Zi towards the wearer's skin and an outward direction Z. away from the wearer's skin. Although in the depicted embodiment, both the outer cover 100 and the insert 200 are substantially symmetrical around a longitudinal central axis Y1, alternative embodiments thereof may be non-symmetrical.

As depicted in FIG. 1, in the absorbent article 1, the insert 200 is detachably attached to the outer cover 100, such that the insert 200 covers an inward facing side of said outer cover 100, and the outer cover 100 forms an outermost surface of the absorbent article 1. Thus an innermost surface of the outer cover 100 faces an outermost surface of the insert 200, such that for the opposite surfaces holds that, during use, an outward facing surface of the outer cover 100 is intended to face away from the wearer and the inward facing surface of the insert 200 contacts the wearer's skin. In the depicted absorbent article 1, a substantial part of both the innermost surface of the outer cover 100 and the innermost surface of the insert 200 are visible, together forming the innermost surface of the absorbent article that contacts the wearer's skin during use.

The absorbent article 1 has a front section aSf, a back section aSb, and a crotch section aSc provided there between along the longitudinal central axis Y1, together spanning a total longitudinal length aL measured along said longitudinal central axis Y1, from a front edge of the absorbent article 1 to a back edge of the absorbent article 1, via the crotch section Sc. As such, the total longitudinal length aL is the maximum longitudinal length of the absorbent article 1 as measured over a minimum width of the crotch section aSc of the absorbent article 1.

Similarly, the outer cover 100 has a front section oSf, a back section oSb, and a crotch section oSc provided therebetween along the longitudinal central axis Ya (see FIG. 3a). The total longitudinal length oL of the outer cover 100 is the maximum length from a front edge 101 of the outer cover 100 to a back edge 102 of the outer cover, as measured over a minimum width of the crotch section oSc of the outer cover 100. Accordingly, portions of the outer cover 100 extending transversally beyond the minimum width of the crotch section oSc of the outer cover are disregarded when determining the longitudinal length oLc of the outer cover 100.

The longitudinal length aL of the absorbent article 1 may correspond approximately to the longitudinal length oL of the outer cover 100. However, it is to be understood that the presence of the absorbent insert 200 attached to the outer cover 100 in the absorbent article may imply that the outer cover is e.g. wrinkled or slightly contracted in the longitudinal direction. Accordingly, the longitudinal length aL of the absorbent article 1 may be less than the longitudinal length oL of the outer cover 100.

The crotch width aWc of the absorbent article 1 is the minimum width of the crotch section aSc of the absorbent article 1, i.e. the minimum width of the portion of the absorbent article intended to be positioned between the leg openings formed by the absorbent article when in use. In the absorbent article, this may thus be the minimum width of the crotch section of either one or both of the insert 200 and the outer cover 100. Similarly, the crotch width oWc of the outer cover 100 is the minimum width of the crotch section oSc of the outer cover 100.

### Outer Cover-General

The crotch section oSc of the outer cover 100 interconnects the front and back sections oSf, oSb, of the outer cover together being shaped to form a pant-like structure when the first and second side portions 110, 111 and front section Sf are attached to one another.

The outer cover 100 comprises a garment portion 150, that forms the main structure, i.e. the front, back and crotch sections of the outer cover. For example, and as in the illustrated embodiment, the garment portion 150 may comprise a main body 109, forming a transversally central portion of the garment portion 150, and extending longitudinally from the back edge 102 of the outer cover, via the crotch portion oSc and to the front edge 101 of the outer cover. As such, the crotch section oSc of the outer cover, at least a central portion of the front section oSf of the outer cover, and at least a central portion of the back section of the outer cover oSb may be formed by the main body 109. In the front and/or back section of the outer cover, the main body of the outer garment 150 may be provided with the one or more transversally extending side portions 110, 111.

In the back section oSb, the outer cover 100 may comprise first and second side portions 110, 111 for enabling attachment of the outer cover 100 around the waist of a wearer. In the illustrated embodiment, only the back section oSb is provided with first and second side portions, adapted to be fastened to the front section oSf of the outer cover 100. However, in other embodiments, the front section oSf may be provided with side portions, and/or the front section oSf and the back section oSb may be provided with side portions. However, the illustrated variant with the back section oSb having first and second side portions 100, 111 is believed to be advantageous for facilitating use by a caregiver and enhancing comfort for the wearer.

The side portions 110, 111 in the back section oSb may, as in the illustrated embodiments, extend, substantially symmetrical, outward from the back edge 102 in transverse direction x, and are each provided with attachment means 115, 116 on the inward facing side, at a respective distal end thereof. The attachment means 115, 116 are adapted to detachably attach to matching surfaces (117) on the outward facing side of the front section oSf of the outer cover 100. Said attachment means 115, 116 and matching surfaces may for example consist of hook and loop surfaces.

In the illustrated variant, a total transversal width between a distal edge of each of the side portions 110, 111 is larger than a total transversal width of the front edge 101 of the outer cover 100, such that the side portions clearly indicate the back edge 102 of the outer cover 100 and can be reached around to attach to the front section Sf of the outer cover 100 for forming the pant-like structure. The closure in the front allows for a wearer lying down when being changed by a caregiver. The total transversal width spanned by the side portions 110, 111 and the total transversal width of the front edge 101 are chosen to fit a predetermined waist size of a person and the absorbent article 100 may be made available for a number of size ranges.

It will be obvious to the skilled person that the same effects can be achieved with side portions 110, 111 that are not symmetrically extending from the back edge 102, but instead have a first transversal width of one side portion be different from a second transversal width of another side portion.

The outer cover is configured to be washable, preferably machine-washable, as discussed in the Summary section of this application.

As also discussed in the Summary section of this application, the garment portion 150 may be made from a breathable, liquid permeable material, such as a textile material, enabling the garment portion 150 to look and feel similar to regular underwear. The garment portion may comprise one or more panels, that together form at least 80%, preferably at least 90%, of the surface area of the outer cover 100. Suitable materials for the garment portion 150 may comprise or consist of one or more of cotton, viscose, polyamide and polyester. Use of polyester materials is preferred when the outer cover needs to be washable, since such materials are known to retain their original size and shape better. To ensure a snug fit, independent of exact body-shape and throughout a wearer's movements, the garment portion 150 preferably is made of stretchable material, having elastic properties in the longitudinal direction Y and/or the transverse direction X. Hereto, the material may be either a woven or knitted textile, with knitted textiles being preferred due to having better elastic properties.

As such, the garment portion 150 may be made by one or more fabric panels. For example, one fabric panel may form each out of the main portion 109 and the side portions 110, 111.

To ensure the outer cover 100 staying in place in the crotch section oSc when the absorbent article 1 is worn, outer leg elastic members 151, 152 may, as in the illustrated embodiment, be provided at the outer edges of the crotch section oSc of the outer cover 100, for securing the outer cover in place around the legs of a wearer. Through the garment portion 150 of the outer cover 100 being made from a breathable material, these outer leg elastic members 151, 152 do not provide fluid tightness and thus act not as fluid barriers. The outer leg elastic members 151, 152 may be elastic wire, cord, strip, thread, etc, that is attached to the edges of the garment portion 150 in the crotch section oSc or interposed in between layers of material that form said edges of the garment portion 150, by sewing, gluing, thermal welding, etc.

The outer cover 100 is made from a breathable, liquid permeable material that provides the absorbent article with an undergarment shape, allowing the article being worn by a wearer similar to underpants whilst enabling easy changing of said wearer by a care-taker. This particular material used resembles regular underpants more closely than the backsheet material of diapers normally forming the outermost surface thereof, making this particular absorbent article more comfortable to wear and giving the wearer an increased feeling of dignity. Thus the outer cover 100 holds the insert in place, while the insert 200 provides the absorbent article 1 with leakage security and the absorptive capacity for retaining liquid and fecal material, i.e. the outer cover 100 has no fluid barriers.

### Insert-General

It will occur to the skilled person that, since the outer cover 100 is reusable, it may be teamed with various different inserts, that may have features that look or are different from the ones depicted in FIGS. 2a-2c. Some non-limiting, possible variations are already indicated in this description. The depicted insert 200 has a liquid-permeable topsheet 205, a liquid-impermeable backsheet 206, an absorbent assembly 230, inner side leakage barriers 220, 221, insert leg elastic members 210, 211 and a set of cover fastener areas 221, 222 arranged in the front section iSf of the insert, and another set of cover fastener areas 241, 242 arranged in the back section iSb of the insert.

The insert 200 is extended along its longitudinal direction Y and transverse direction X. The topsheet 205 forms part of the innermost surface of the insert 200 facing in an inward direction Zi and which is intended to be directed towards the wearer when the absorbent article is worn. The backsheet 206 is forming at least part of the outermost surface of the insert 200, facing in an outward direction Zo. The absorbent assembly 230 is enclosed between the topsheet 205 and the backsheet 206. The backsheet may cover the entire surface area of the insert 200, that is the backsheet forms the outermost surface of the insert 200. Thus the width iW and the length iL of the insert 200 maybe equal to the width and length of the backsheet.

The liquid-permeable topsheet 205 can consist of any material that is suitable for the purpose. Examples of commonly encountered liquid-permeable topsheet materials are non-woven textile materials, known as nonwoven materials, perforated plastic films, meshes made of plastic or textile, and liquid-permeable foam layers. Liquid-permeable covering materials which consist of continuous thin fibers that extend predominantly in the longitudinal or transverse direction of the article are also encountered. Laminates consisting of two or more of the above-mentioned possible covering materials are also commonly encountered, as are coverings consisting of different materials in different parts of the surface.

The liquid-impermeable backsheet 206 may extend beyond the absorbent assembly 230 around the periphery of the entire absorbent assembly 230. The backsheet can consist of a range of different materials. The backsheet may consist of a thin and liquid-tight plastic film, although it is also possible to use other types of liquid-tight material such as nonwoven materials that have been made liquid-tight, for example by means of plastic coating, liquid-tight foam layers, liquid-tight adhesive or similar. The backsheet can also consist of a liquid-tight, vapor-permeable material known as a breathable material.

The innermost layer of the insert 100 comprising the liquid-permeable topsheet 205 is attached to the backsheet 206 outside the absorbent assembly 230 along the entire periphery of the absorbent assembly 230. The innermost layer comprising the liquid-permeable topsheet 205 and the backsheet may be attached to one another by a number of different means. Examples of means of attachment include gluing, thermal fusion, ultrasonic welding or the like.

The absorbent assembly 230 may, as depicted, have a length and width that are smaller than the innermost layer comprising the topsheet 205 and the backsheet 206, thus being present in a central region of the insert 200 where liquid and feces exit the body of the wearer. This results in a less bulky, more comfortable design, thereby being preferred. However, it will occur to the skilled person that alternative sizes, shapes and configurations of the absorbent assembly 230 may also be suitable for use in an absorbent article as presented herein. As illustrated in FIG. 2a, the absorbent assembly 230 may have a longitudinal length iLc.

For example, and as illustrated in the drawings, the absorbent assembly 230 may have an hourglass-shape, which is intended to be situated at the narrowest area between the wearer's thighs when being worn. The longitudinal side edges 203, 204 of the innermost layer comprising the topsheet 205 and of the backsheet 206 also correspond to this hour-glass shape. This results in the insert being more formfitting and thus more comfortable to wear than if it were to have a rectangular shape.

The absorbent assembly 230 is depicted (FIGS. 2a-2c) as having a first layer 231, a second layer 232 and a receiving layer 233. The receiving layer 233 is positioned directly below the topsheet 205 and is adapted to rapidly receive quite large quantities of liquid and to retain this liquid temporarily, in order subsequently to release the temporarily stored liquid to other parts of the absorbent assembly 230. Alternative configurations of the absorbent assembly 230 may be envisaged and both the second layer 232 and the receiving layer 233 may be considered optional, independent from one another. The absorbent assembly as such may be constructed similarly to known absorbent assemblies and as such can be constructed from one or more layers of cellulose fluff pulp. The cellulose fluff pulp can be mixed for this purpose with fibers or particles of a high-absorbency polymer material, known as superabsorbents, of the kind which, in conjunction with absorption, chemically bonds large quantities of liquid to form a liquid-containing gel. The absorbent assembly 230 can also contain high-absorbency polymer material arranged in a layer inside the absorption body or in conjunction with the surface or surfaces of the absorption body. Additional components to improve the characteristics of the absorbent assembly 230 can also be present in the absorbent assembly 230. Examples of such components include binding fibers, different types of liquid-distributing layers or fibers, form-stabilizing components, reinforcing fibers or the like. The absorbent assembly 230 can naturally also consist of other types of absorption material, such as absorbent nonwoven material, absorbent foam, textile materials, peat or mixtures of different kinds of absorption material.

### Insert Leg Elastic Members

The insert leg elastic members 210, 211, as depicted in FIGS. 2a to 2c, are arranged in between the longitudinal outer edges of the absorbent assembly 230 and the longitudinal outer edges 203, 204 of the insert 200 on both sides of the insert 200, running essentially in the longitudinal direction of the insert 200. As depicted in FIG. 2a, the insert leg elastic members 210, 211 may be partially curved. For example, the insert leg elastic members may be partially curved so as to follow the shape of the absorbent assembly 230, when the absorbent assembly is hour-glass shaped. A maximum transversal width over the insert leg elastic members 201, 211 is defined as the leg elastics width iWI. As illustrated in the Figures, the transversal width over the insert leg elastic members 210, 211 is the width measured in a transversal direction including both insert leg elastic members 210, 211 in the pair of insert leg elastic members 210. 211. The transversal width over the insert leg elastic members 210, 211 may, as in the illustrated embodiment, vary along the length of the insert leg elastic members 210, 211. The maximum transversal width over the leg elastic members 210, 211 in each of the front section iSf or back section iSb of the insert is the maximum transversal width which may be found along the length of the insert leg elastic members 210, 211 in said front or back section iSf, iSb, respectively.

The insert leg elastic members 210, 211 are included in the insert 200 for preventing liquid and faeces from leaking out past the longitudinal edges of the insert, ensuring the insert 200 is leakproof and allowing the entire outer cover 100 being made of liquid-permeable material. Each of the insert leg elastic members 210, 211 may consist of one or more elastic threads that have been applied in their stretched state between the innermost layer comprising the liquid-permeable topsheet 205 and the backsheet. The pair of insert leg elastics members have a longitudinal leg elastics length iLl, which may extend along at least 60% of the length of the insert. For example, the leg elastics length may be approximately coinciding with the length iLc of the absorbent assembly 230. The elastic members 210, 211 may be attached to the backsheet and the topsheet 205 by gluing, ultrasonic welding or the like.

As illustrated in FIG. 2a, in each of the front section iSf and the back section iSb of the insert 100, the insert 100 has a maximum width iW, and the leg elastics width iWI being the maximum transversal width between the leg elastic members 210, 211 is smaller than the maximum width Wi of the insert and larger than a transversal maximum width of the absorbent assembly 230. A leg edge portion 212, 213 is defined between each insert leg elastic member 210, 211 and the adjacent side edge 203, 204 of the insert 200, in each of the front and back sections iSf, iSb of the insert 200. The leg edge portions 212, 213, may have a maximum width being at least 1 cm, such as at least 2 cm, and provide additional fluid leakage protection as well as increased comfort to the wearer.

### Insert Inner Side Leakage Barriers

In order further to prevent liquid or faeces from leaking out via the side edges of the insert 200, the insert 200 may be provided with inner side leakage barriers 220, 221 on the inner surface, as depicted in FIGS. 2a and 2b.

The inner side leakage barriers 260, 261 are arranged on either side of and adjacent to the longitudinal edges of the absorbent assembly 230, and they extend essentially in the longitudinal direction of the insert 200. The inner side leakage barriers 260, 261 comprise raisable parts which, when the insert is in use, raise in an inward direction from the topsheet 205, so as to form barriers against side leakage. The raisable parts of the inner side leakage barriers 260, 261 extend at least over a part of the crotch section iSc of the insert 200.

A maximum transversal barrier width iWg is measured between the attached edges 260b, 261b of the raisable parts of the inner side leakage barriers 260, 261. The maximum transversal barrier width iWg may be smaller than a maximum transversal width iWI over the insert leg elastic members 210, 211, if present, and optionally larger than a maximum transversal width of the absorbent assembly 230. The respective inner side leakage barrier 260; 261 may be executed from a separate material strip, which exhibits two essentially parallel longitudinal edges. The material strip may be double-folded, in conjunction with which the parallel longitudinal edges are arranged adjacent to one another. At least a longitudinal edge of each material strip is attached to the innermost layer comprising the topsheet 205 and constitutes the attached edge 260b, 261b of the respective inner side leakage barrier 260, 261. The folded edge of the material strip constitutes the free edge of the inner side leakage barrier 260; 261. The inner side leakage barriers 260, 261 are folded down over the innermost layer during manufacturing and storage, and have a raised configuration during use, in which at least in the crotch section iSc of the insert, the free edge is lifted up from the innermost layer comprising the topsheet 205.

In the depicted insert 200, the longitudinal ends of the material strips are attached to the innermost layer comprising the topsheet 205 on the front edge 201 and the back edge 202 of the insert 200.

The inner side leakage barriers 260, 261 further comprise elastic elements 260a, 261a attached to the inner side leakage barriers 260, 261 in a pre-tensioned state. The elastic elements 260a, 261a are conveniently arranged adjacent to the free edges of the inner side leakage barriers 260, 261. When the pre-tensioned elastic elements 260a, 261a are released, they contract together with the free edges of the inner side leakage barriers 260, 261, thereby causing the inner side leakage barriers 260, 261 to be brought into a raised configuration remote from the liquid-permeable topsheet 205. For example, the inner side leakage barriers 260, 261 may be brought into a raised position at least in crotch part of the insert corresponding to at least 60% of the longitudinal length of the insert, the crotch part being spaced from both the front end 201 and the back end 202 of the insert.

As depicted in FIG. 3a, the inner side leakage barriers 260, 261 form raisable parts where the inner side leakage barriers 260, 261 may be brought to the raised configuration over a majority of the longitudinal length of the insert 200. As depicted in FIG. 3b, the inner side leakage barriers 260, 261 may be attached to the topsheet 205 in attached regions adjacent the front end and the back end, respectively of the insert. In said attached regions, the material strip forming each inner side leakage barrier 260, 261 is attached to the topsheet, e.g. via a weld or an adhesive, such that, in said attached regions, the inner side leakage barriers are not raisable. For example, the inner side leakage barriers 260, 261 may be attached to the inward facing side of the insert 200 in the front edge portion 201′ and/or in the rear edge portion 202′ of the insert.

Alternatively, the longitudinal ends of the material strips may be unattached to the innermost layer, such that the free edges of the material strips forming the inner side leakage barriers 220, 221 are free to raise up from the innermost layer upon bending of the insert into the use position, eventually without the help from pre-tensioned elastic elements.

To reliably and comfortably hold the insert 200 in the outer cover 100, forming the absorbent article 1, the insert 200 and outer cover 100 are detachably attachable to one another.

### Cover Fastener Areas of Insert

Hereto, in the depicted exemplary embodiments in FIGS. 1, 2a to 3b, and the insert 200 comprises a set of cover fastener areas 241, 242 in the back section iSb and another set of cover fastener areas 221, 222 in the front section iSf.

Each cover fastener area 221, 222, 241, 242 comprises a surface fastening material for providing detachable attachment of the insert to the outer cover.

In the illustrated variant, the set of cover fastener areas in the back section iSb and the set of cover fastener areas in the front section iSf each comprises two transversally spaced cover fastener areas 241, 242; 221, 222. However, other variants are possible, such that at least one of the sets of cover fastener areas comprising only one single cover fastener area, or at least one of the sets of cover fastener areas comprising more than two cover fastener areas.

Regardless of the amount of cover fastener areas comprised in a set, a transversal fastening width iWf may be defined as the transversal width spanned by the set of one or more cover fastener areas.

In the illustrated variant, each one of the cover fastener areas in the set is arranged at the same longitudinal location, and each one of the cover fastener areas moreover have the same length iAl as seen in the longitudinal direction. However, other variants are possible, wherein for example at least one cover fastener area in a set is arranged at a different longitudinal location than another cover fastener area in the set. Still, the set of one or more cover fastener areas spans a longitudinal length iAl along the longitudinal axis L.

A front edge portion 201′ and/or a back edge portion 202′ is/are defined as the portion of the insert 200 extending from the front edge 201 or back edge 202, respectively, of the insert 200 and over the longitudinal length iAl spanned by the set of one or more cover fastener areas 221, 222; 241, 242 in the front section iSf or back section iSb, respectively.

Each one of the front edge portion 201′ and/or the back edge portion 202′ may extend over an edge portion length being for example less than 20% of the longitudinal length of the insert, such as less than 10% of the longitudinal length of the insert.

As such, it may be ensured a large portion of the inward facing surface of the insert 200 remains available for directly contacting the wearer's genital area and thus limiting the risk of soiling of the outer cover 100. As exemplified in the illustrated embodiment, in the longitudinal direction Y, each of the detachable cover fastener areas 221, 222, 241, 242 in the set may be positioned at, i.e. be directly adjacent, the front edge 201 or the back edge 202, respectively. This is an advantage in manufacturing, as one piece of a fastener member material may be arranged over two subsequent inserts when formed on a continuous insert material line. In this instance, one cutting operation will separate the piece of fastener member material into two at the same time as the continuous insert material line is cut into separate inserts. In this case, the longitudinal length iAl spanned by the set of cover fastener areas will correspond to the longitudinal length of the respective edge portion 201′, 202′ of the insert 200. For example, each set of one or more cover fastener areas 221, 222; 241, 242 may span a longitudinal length iAl being less than 20% of the longitudinal length of the insert, such as less than 10% of the longitudinal length of the insert.

In the transverse direction X, each set of cover fastener areas 221, 222; 241, 242 in the front and back section iSf, iSb, respectively, extends over a transversal fastening width iWf. As exemplified in the drawings, the transversal fastening width iWf may be no less than, and preferably greater than, the leg elastics width iWI.

This particular positioning allows for the insert to be sufficiently tensioned when the absorbent article 1 is put on a wearer, ensuring reliable functioning of the insert leg elastic members 210, 211.

The insert leg elastic members extend along a longitudinal insert leg elastics length. The front edge portion 201′ and the back edge portion 202′ may advantageously be arranged so as not to overlap with the longitudinal insert leg elastics length. In other words, each set of one or more cover fastener areas is confined to a front edge portion 201′ or a back edge portion 202′ not spanned by the leg elastics length LI.

The transversal fastening width iWf may, as in the illustrated embodiment, extend over at least part of the leg edge portions 212, 213 of the insert, so as to ensure that the leg portions are automatically folded outward when the insert is attached to an outer cover. For example, the transversal fastening width may span into at least 50% of the transversal width of each leg portion, or at least 80% of the transversal width of each leg portion.

As in the illustrated example, in the back section iSb and/or the front section iSf of the insert 200, the set of one or more cover fastener areas may comprise a first subset of one or more cover fastener areas 221; 241, and a second subset of one or more cover fastener areas 222; 242. In the back section iSb, the first subset of one or more cover fastener areas 241 is spaced from the second subset of one or more cover fastener areas 242 by a transversal spacing width iWs.

Similarly, in the front section iSf, the first subset of one or more cover fastener areas 221 is spaced from the second subset of one or more cover fastener areas 222 by a transversal spacing width iWs. As such, in the front section iSf as well as in the back section iSb, the insert 200 is free from cover fastener areas over a spacing width iWs as measured between the subsets in the front or back section, respectively.

This contributes to the correct shaping of the insert in use, since the insert is to be unattached to the outer cover over the transversal spacing width. Moreover, by providing cover fastener areas substantially only at the locations which are most critical to ensure the correct positioning of the insert in the outer cover, correct assembly of the insert and an outer cover may be facilitated.

The transversal spacing width may for example be at least 70 % of the transversal fastening width.

In the illustrated embodiment, each subset of one or more cover fastener areas comprises one single cover fastener area. As such, the first and second subsets of one or more cover fastener areas together form a pair comprising two cover fastener areas (one cover fastener area in each subset) being spaced by the spacing width iWs.

As illustrated in the examples of the Figures, the cover fastener areas 221, 222, 241, 242 may be provided on the innermost layer comprising the topsheet 205, each at a respective corner of said innermost layer comprising the topsheet 205, for detachably attaching the insert 200 to the outer cover 100. The cover fastener areas 221, 222, 241, 242 are located outside the crotch section iSc, thereby preventing any overlap of the attachment to the outer cover 100 with the crotch section oSc and thus reducing the risk of soiling of said outer cover. The cover fastener areas 221, 222, 241, 242 together may cover less than 5% of the total surface area of the innermost layer comprising the topsheet 205. This is found to be sufficient for reliably attaching the insert inside the outer cover 100, thus using more material would result in unnecessary waste. Moreover, this implies that a relatively small portion of the innermost layer of the insert is occupied by the cover fastener areas 221, 222, 241, 242, and hence a relatively large portion of the innermost layer of the insert may be available for receiving bodily exudates when the absorbent article 1 is in use.

As mentioned in the above each of the cover fastener areas 221, 222, 241, 242 comprises a surface fastening material. For example, and as illustrated herein, the surface fastening material provides frictional and/or mechanical attachment. For example, the surface fastening material may be a surface fastening material comprising a plurality of discrete elements for providing frictional and/or mechanical attachment. A well-known type of such a surface fastening material is loop material, which is compatible with hook material, together also commonly known as Velcro©.

In the absorbent article 1 (for example as depicted in FIG. 1), the cover fastener areas 221, 222, 241, 242 are attached to the outer cover 100 in front and back flap members 120, 140.

### Flap Member of Outer Cover

As seen in FIGS. 1 and 3a, in each one of the front section oSf and the back section oSb of the outer cover 100, a flap member 120, 140 is arranged so as to extend from the garment portion 150. The outward facing side of the flap members, in a use position, is facing a space which is open in a longitudinal direction Y towards the crotch section Sc for receiving the respective back edge portion 202′ or a front edge portion 201′ of the insert 200 therein, while the insert 200 extends over at least part of the crotch section Sc of the absorbent article 1. The inward facing side of the insert 200 is to be attached to the outer cover 100 via the front and back flap members 120, 140.

In the outer cover 100, the front and back flap members 120, 140 may be made from the same material as the garment portion 150. In particular, a single sheet of material may form at least one layer of the front and back flap members 120, 140, and the garment portion 150.

Having a flap member 120, 140 in both the back and the front section is advantageous, as it allows the insert being fully secured over the crotch area, ensuring good positioning throughout use. However, depending on the particular material and configuration choices for the outer cover and insert, having only one flap member present in the front or back section may suffice.

In the illustrated variant, a single flap member 120, 140 is arranged in each of the back and front section oSb, oSf.

In some variants, the flap member 120, 140 may extend from the garment portion 150 via e.g. a fold. For example, the flap member 120, 140 may comprise a fabric panel also forming at least part of the main portion 109 of the garment portion 150.

In other variants, as in the illustrated variant, the flap member 120, 140 may be attached to the garment portion 150 by a joint, such as e.g. seams or stitches. For example, the flap member may comprise a fabric panel being attached to the garment portion.

As in the illustrated variant, the flap member 120, 140 may extend from the front edge 101 or back edge 102, respectively, of the outer cover 100.

For example, the flap member 120, 140 may be joined (either via a fold or via joint members such as e.g. seams or stitches) to the garment portion 150 along at least one out of a back or front edge, respectively, and/or a pair of side edges.

In the depicted configuration shown in FIG. 3a, the front and back flap members 120, 140 each extend transversally between a pair of side joints 148, 149; 128, 129, joining an inward facing surface of the garment portion 150 of the outer cover 100 with the flap member. Thus, the side joints join the side edges of the respective front and back flap members 120, 140 to the garment portion 150 such that the flap members are automatically in a closed position that forms the use position. However, to make details on the inside flap member more easily visible, the sides are unattached in the outer cover 100 as depicted in FIG. 3b, such that both the front and back flap members 120, 140 are shown in an open position, making the insides thereof, consisting of a body facing and a garment facing side, visible.

Through the presence of the joints 148, 149, 128, 129, ensures the front and back flap members 120, 140 are held towards correct configuration for use, thereby protecting a wearer's skin from contacting any insert attachment means inside the flap members. Further, the presence of the joints 148, 149, 128, 129 ensures that transversal forces to the outer cover as introduced when the outer cover is closed around the waist of a wearer are transmitted to the flap member. Accordingly, the flap member 120, 140 may be stretched out in a transversal direction.

In other variants of an outer cover, the flap member or flap members may be connected to the garment portion 150 via e.g. only side joints or only a rear joint or fold.

When the flap member 120, 130 extends in a transversal direction X between a first and a second side joint 128, 129; 148, 149 joining the flap member to the garment portion 150, the space being open in a longitudinal direction towards the crotch section has a space width oWt as defined between the first and second side joints 128, 129; 148, 149. The space width oWt of each respective flap member 120, 140, is equal to or larger than the width of the respective front or back edge 201, 202 of the insert 200. The space width oWt of the back flap member 140 may be substantially equal to the space width of the front flap member 120. In the depicted outer cover 200, the front flap member 120 has a space width substantially equal to the width of said front section. In the back section Sb, the space width of the flap member 140 is such that the first and second sides are attached to a respective edge of the first and second side portions 110, 111 that is nearest to the longitudinal central axis Y1. Advantageously, this configuration allows the joints 148, 149 of the back flap member 140 being attached at or with the seam that attaches one or more material layers of the first and second side portions 110, 111 to the garment portion 150.

Flat seams may be used, to further reduce the risk of skin chafing for the wearer.

### Insert Fastener Areas of the Outer Cover

For example, as in the illustrated variant, for detachably fastening the insert 200 to the outer cover 100, in each of the front section Sf and the back section Sb, the outer cover may comprise a flap member, wherein each flap member is provided with a set of one or more insert fastener areas 121, 122; 141, 142 on an outer surface, i.e. a surface that is adjoining the space formed by the respective flap member when in the use position. In the depicted outer cover, in the front section oSf, the front flap member 120 is provided with a set comprising two insert fastener areas 121, 122 on an outward facing side 120a of the flap member 120. Similarly, in the back section Sb, the back flap member 140 is provided with a set comprising two insert fastener areas 141, 142 on an outward facing inner side 120a of the flap member. A longitudinal length of each of the insert fastener areas 121, 122, 141, 142 is smaller than a longitudinal length of a respective front or back flap member 120, 140 in closed position, i.e. a longitudinal length of an outward facing inner side 120a, 140a of the flap members 120, 140.

Thus, as in the illustrated embodiment, each insert fastener area 121, 122, 141, 142 may be provided by a corresponding insert fastener member 121a, 122a, 141a, 142a, as shown in FIG. 3b. As such, each flap member 120, 140 comprises a fabric panel to which the insert fastener members 121a, 122a; 141a, 142a providing the insert fastener areas are attached.

Preferably, and as in the illustrated version, the insert fastener members of the flap member are completely confined within the area of the fabric panel.

As in the illustrated embodiment, the flap member 120, 140 may have a maximum longitudinal length oLt being less than 20% of the longitudinal length of the outer cover. For example, a sum of the maximum longitudinal lengths of the front flap member 120 and the back flap member 140 may bel ss than 30% of the longitudinal length of the outer cover. Thus, the attachment of the insert to the outer cover takes up only a minority of the length of the outer cover.

The outward facing side of each flap member 120, 140 comprises a set of one or more insert fastener areas 121, 122; 141, 142, each insert fastener area comprising a surface fastening material for providing detachable attachment to the insert.

Each set of insert fastener areas spans a transversal fastening width oWf. The transversal fastening width oWf may preferably be no less than a minimum transversal width oWc of the outer cover, i.e. no less than the crotch width oWc of the outer cover. As such, the transversal fastening width oWf will be no less than a minimum leg elastics spacing width as measured between the leg elastics 151, 152 of the outer cover 100.

The transversal fastening width oWf may preferably be less than a maximum leg elastics spacing width oWI as measured between the leg elastics 151, 152 of the outer cover.

As such, it may be ensured that, when an insert 200 is attached to the outer cover 100, the leg elastics 152, 152 of the outer cover assists in providing a correct position for the insert visavi the wearer.

Moreover, it may be ensured that the outer cover, when used, provides the required tension in tranversal direction to the insert so as to stretch the insert leg elastic members 210, 211 (and inner side barriers 220, 221) to their correct position, when in the insert 200 is as described in the above.

For example, and as in the illustrated variant, the set of insert fastener areas may comprise a first subset of insert fastener areas 121; 141 and a second subset of insert fastener areas 122; 142, the first and seconds subsets being transversally spaced by a transversal spacing width oWs. To further ensure correct positioning and shaping of the insert visavi the outer cover, the transversal fastener spacing width oWs may be no less than the minimum transversal width oWc of the outer cover.

In the illustrated variant, each subset of insert fastener areas comprises one single insert fastener area.

### Absorbent Article

In view of the above description of a variant of an absorbent insert 200 and an outer cover 100, variant an absorbent article 1 as depicted in FIG. 1 is also described.

The absorbent article 1 extends along a longitudinal direction Y, and along a transversal direction X, which is perpendicular to the longitudinal direction Y, and wherein a thickness direction Z is defined perpendicular to both the longitudinal and transversal directions X, Y, said thickness direction being an inward direction Zi towards the wearer when the article is worn, and an outward direction Zo away from the wearer when the article is worn, the absorbent article comprising a front section Sf, a back section Sb and a crotch section Sc extending between the front section Sf and the back section Sb along the longitudinal direction Y, the absorbent article 1 comprising: a reusable outer cover 100, comprising a garment portion 150 that extends in at least the front section Sf, and the back section Sb of the absorbent article 1, said garment portion 150 being adapted to fit around a wearer's waist, with the front section Sf being positioned at or close to the wearer's belly side and the back section Sb being positioned against the wearer's lower back when the article is worn, and an absorbent insert 200 comprising a liquid impermeable backsheet, a liquid permeable topsheet 205, and an absorbent assembly 230 provided in between said backsheet and said topsheet, the insert 200 being shaped to, in use, cover a wearer's pubic area and a predefined part of the buttocks with an inward facing side comprising said topsheet directed towards the wearer, and an outward facing side directed towards the outer cover 100.

The described absorbent article 1 is an example of an absorbent article wherein, in at least one, preferably each, of the front section Sf and the back section Sb of the absorbent article, the outer cover comprises a flap member 120, 140 extending from the garment portion 150 with an outward facing inner side 120a, 140a of the flap member 120, 140 facing the inward facing side of the insert 200 in a front or back edge portion 201′, 202′, respectively of the insert. Further, in said at least one, preferably each, of the front section Sf and the back section Sb of the absorbent article, the absorbent article comprises a set of one or more attachment areas 21, 22; 41, 42, wherein, in each attachment area, a surface area of the inward facing side of the insert 200 is detachably attached to a surface area of said outward facing side of the flap member 120, 140 via at least one surface fastening material, such that the front or back edge portion 201′, 202′, respectively, of the insert 200 is detachably attached to the outer cover 100 in said set of attachment areas 21, 22; 41, 42 while, in at least said crotch section Sc of the absorbent article 1, the inward facing side of the insert 200 forms the innermost side of the absorbent article 1, so as to be directly adjacent the wearer's skin, when the article is in use.

Accordingly, the article presents the advantages obtained by the attachment between the outer cover and the insert taking place via the outward facing inner side of the flap member and the inward facing side of the insert, i.e. advantages contributing to a correct and secure position of the insert in relation to the wearer when the absorbent article is worn.

Moreover, the described absorbent article 1 is an example of an absorbent article wherein the insert 200 extends longitudinally between a front edge 201 and a back edge 202, and comprises longitudinal side edges 203, 204 connecting the front edge 201 and the back edge 202, and the insert 200 comprises a pair of insert leg elastic members 210, 211 extending essentially along the longitudinal direction Y of the insert, and arranged on both sides of the insert, between the absorbent assembly 230 and the longitudinal side edges 203, 204 of the insert, wherein a leg elastics length iLl is defined as the longitudinal length along said insert leg elastic members 210, 211, and wherein, in each out of the back section aSb and the front section aSf, a leg elastics width iWI is defined as the maximum transversal width over said insert leg elastic members 210, 211 in said front or back section (aSf, aSb), respectively. In at least one, preferably each, of the front section aSf and the back section aSb of the absorbent article, the absorbent article comprises a set of one or more attachment areas 21, 22; 41, 42 for attaching a front or back edge portion 201′, 202′, respectively of the insert to the outer cover, wherein, in each attachment area, a surface area of the inward facing side or the outward facing side of the insert 200 is detachably attached to a surface area of the outer cover 100 via at least one surface fastening material. Further, a transversal fastening width aWf, being the transversal width spanned by the set of one or more attachment areas 21, 22; 41, 42, is no less than the leg elastics width iWI in said front or back section aSf, aSb, respectively, such that the front or back edge portion 201′, 202′, respectively, of the insert 200 is detachably attached to the outer cover 100 in said set of attachment areas 21, 22; 41, 42 while, in at least said crotch section aSc of the absorbent article 1, the inward facing side of the insert 200 forms the innermost side of the absorbent article 1, so as to be directly adjacent the wearer's skin, when the article is in use.

Accordingly, the article presents the advantages obtained by the set of attachment areas having a transversal fastening width being no less than the leg elastics width, such that the leg elastics of the insert are stretched when the article is in use, i.e. advantages contributing to a correct and secure position of the insert in relation to the wearer when the absorbent article is worn.

The advantages obtained by the attachment between the outer cover and the insert taking place via the outward facing inner side of the flap member and the inward facing side of the insert, and the advantages obtained by the set of attachment areas having a transversal fastening width being no less than the leg elastics width may be achieved one by one. However, as in the illustrated variant, a combination of the features resulting in a combination of the advantages may be particularly preferred. For example, when the leg elastic members are stretched out by virtue of the transversal fastening width being no less than the leg elastics width, the attachment areas may have an increased need for being protected from tearing or plucking from the outside of the article, when the article is worn, which is met by the attachment taking place via the inward facing side of the insert and the outward facing inner side of the flap member.

It will be understood that the set of attachment areas 21, 22; 41, 42 in the depicted variant of the absorbent article 1 is formed by the insert fastener areas 121, 122; 141, 142 being attached to the cover fastener areas 221, 222; 241, 242 via the surface fastening material of the insert fastener areas being for example a hook material, and the surface fastening material of the cover fastener areas being for example a loop material.

Preferably, the dimensions of the outer cover 100 and the insert 200 are such that, when the insert is normally attached in the outer cover, the front and back flap members 120, 140 do not overlap with the absorbent assembly 230.

For example, each flap member 120, 140 may have a longitudinal flap length oLt being such that, in the use position, when the insert 200 is attached to the outer cover 100, the flap member 120, 140 extend(s) over less than 20% of the insert longitudinal length iL, preferably over less than 10% of the insert longitudinal length iL.

For example, the total area of the front and back flap members 120, 140 may cover less than 80%, preferably less than 85%, of a total insert area. The front and back flap members 120, 140 may be sized such that less than 15% of the total insert area is covered during use, preferably less than 10%.

### Fastener Areas

For example, and as in the illustrated variant, each insert fastener area 121, 122, 141, 142 may be provided by an insert fastener member 121a, 122a, 141a, 142a comprising a fastening material being a hook material. Each insert fastener member may be attached on the outward facing inner side 120a, 140a of the respective front and back flap members 120, 140 of the outer cover.

In the insert, each cover fastener area 221, 222, 242, 242 may be provided by a cover fastener member 221 a. 222 a, 241a, 242a comprising a fastening material being a loop material. Each cover fastener member may be attached on the inward facing side of the insert.

As a result of the placement of the hook members, the hooks thereof are facing away from the wearer's body, when in use, thereby further reducing the risk of chafing of the skin, which is already lower due to the insert fastener areas and cover fastener areas being covered by the flap members.

In the use position, the flap members 120, 140 fully cover the cover fastener areas 221, 222, 241, 242 of the insert 200, thereby also preventing any contact between the wearer's skin and the attachment material. Since such material is often relatively rough, compared to topsheet material and the materials used for the garment portion 150 of the outer cover 100, the covering thereof reduces the risk of chafing.

Although the depicted embodiment shows hook and loop fasteners being used, alternative surface fastening materials may also be suitable and may be used instead of some or all of the insert fastener areas and cover fastener areas. Such alternative surface fastening materials provide substantially uniform fastening over a predetermined surface area, rather than point connections, such as buttons, snaps, rivets, etc, thereby minimizing the risk of chafing to the skin associated with these point connections.

In order to ensure that the outer cover is washable, the surface fastening materials provided thereon should be both reusable and washable.

A further advantage to using fastener areas comprising a surface fastening material, including the depicted hook and loop members, is that the fastening is provided over a surface area, which provides more placement tolerance when attaching the insert to the outer cover. As a result, the attaching of the insert to the outer cover is easier, since it requires less precision, and therefore faster than when point connections are being used.

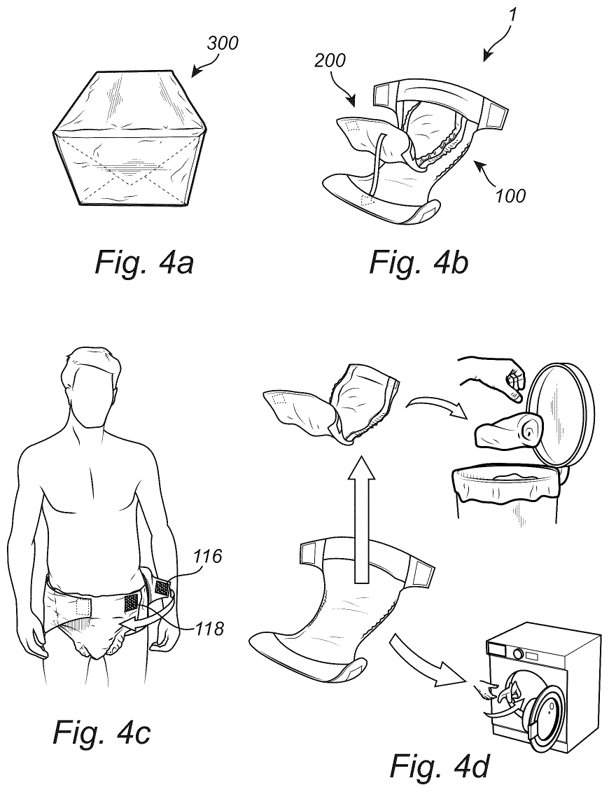

FIGS. 4a, 4b, 4c and 4d depict the use-cycle of an absorbent article 1 similar to that depicted in FIG. 1, respectively showing a package 300 containing at least an insert 200, the attachment of an insert 200 into an outer cover 100, the absorbent article being worn by a wearer and the removal and disposal of an insert 200 and cleaning of the outer cover 1.

The package in FIG. 4a may be available containing a predetermined amount of inserts and/or one or more outer covers. In FIG. 4b is shown that first the insert 200 is detachably attached inside the corresponding flap members of the outer cover 100. Attachment is effected as described in the above. Next, the absorbent article is put on a wearer, as depicted in FIG. 4c. For the particular embodiment of the outer cover 100, side portions 110, 111 are arranged on the back end part of the article and attached to the front end part of the article, on the outward facing surface of the outer cover 100, when the article is put on a wearer. Preferably, the outer cover 100 is provided with special receiving surfaces 117, 118 (See FIG. 4c and FIG. 3a) at the front end part, on the surface facing away from the wearer, as depicted. Said special receiving surfaces 117, 118 are adapted for receiving the attachment means 115, 116 (See FIG. 4c and FIG. 3a) provided on the first and second side portions 110, 111 to ensure good detachable attachment without potentially causing damage to the main material forming the outward facing surface of the outer cover 100.

After use, the entire absorbent article 1 is removed from the wearer by detaching the first and second side portions, followed by separating the insert from the outer cover as depicted in FIG. 4d. In the present embodiment, the insert 200 is a disposable insert, which is thrown away after the use, while the outer cover 100 is washable, such that it may be washed as depicted. In a following use-cycle, the same outer cover 100 is then used with a new, another, insert 200. It will be understood that the described use-cycle will be the same for all absorbent articles according to the invention, independent from the particular shape of the outer cover and the insert. Furthermore, it will be understood that the use-cycle will be substantially the same for absorbent articles according to the present description where the outer cover is non-washable, but reusable, apart from being washed prior to being re-used in the following use-cycle.

The present invention has been described with reference to a number of explanatory embodiments as shown in the figures. Obvious modifications and alternative implementations of some parts or elements will be apparent to those skilled in the art upon reading and understanding the foregoing detailed description. In particular, it will be obvious to the skilled person that various alternatives explicitly mentioned in the detailed description may be implemented independently from other explicitly mentioned alternatives that are not explicitly described as being related thereto. The invention is intended to be construed as encompassing all such modifications and alternative implementations to the extent that they fall within the scope of the appended claims.

1.-94. (canceled)

95. An absorbent article extending along a longitudinal direction, and along a transversal direction, which is perpendicular to the longitudinal direction, and wherein a thickness direction is defined perpendicular to both the longitudinal and transversal directions, said thickness direction being an inward direction towards the wearer when the article is worn, and an outward direction away from the wearer when the article is worn, the absorbent article comprising a front section, a back section and a crotch section extending between the front section and the back section along a longitudinal direction, and comprising:
a reusable outer cover, comprising a garment portion that extends in at least the front section, and the back section of the absorbent article, said garment portion being adapted to fit around a wearer's waist, with the front section being positioned at the wearer's belly side and the back section being positioned against the wearer's lower back when the article is worn; and
an absorbent insert comprising a liquid impermeable backsheet, a liquid permeable topsheet, and an absorbent assembly provided in between said backsheet and said topsheet, the insert being shaped to, in use, cover a wearer's pubic area and a predefined part of the buttocks with an inward facing side comprising said topsheet directed towards the wearer, and an outward facing side directed towards the garment portion of the outer cover,
wherein, in at least one of the front section and the back section of the absorbent article,
the outer cover further comprises at least one flap member extending from said garment portion with an outward facing inner side of the flap member facing the inward facing side of the insert in a front or back edge portion, respectively, of the insert, and
in said at least one of the front section and the back section of the absorbent article, the absorbent article comprises a set of one or more attachment areas, wherein, in each attachment area, a surface area of the inward facing side of the insert is detachably attached to a surface area of said outward facing inner side of the flap member via at least one surface fastening material, such that the front or back edge portion, respectively, of the insert is detachably attached to the outer cover in said set of attachment areas while, in at least said crotch section of the absorbent article, the inward facing side of the insert forms the innermost side of the absorbent article, so as to be directly adjacent the wearer's skin, when the article is in use.

96. The absorbent article according to claim 95, wherein, in said at least one of the front section and the back section of the absorbent article, the outward facing inner side of the flap member of the outer cover comprises a set of one or more insert fastener areas, each insert fastener area comprising a surface fastening material for providing detachable attachment to the inward facing surface of the insert in at least one out of said set of one or more attachment areas, and/or
in said at least one of the front section and the back section of the absorbent article, the inward facing side of the insert comprises a set of one or more cover fastener areas, each cover fastener area comprising a surface fastening material for providing detachable attachment to the outward facing surface of said flap member at least in at least one out of said set of one or more attachment areas.

97. A reusable outer cover for an absorbent article, extending along a longitudinal direction, and along a transversal direction, which is perpendicular to the longitudinal direction, and wherein a thickness direction is defined perpendicular to both the longitudinal and transversal directions, said thickness direction being an inward direction towards the wearer, when the article is worn, and an outward direction away from the wearer, when the article is worn,
the outer cover comprising a garment portion having a longitudinal length that extends along the longitudinal direction, the garment portion being subdivided into a front section, a back section and a crotch section along said longitudinal length, and the garment portion being adapted to fit around a wearer's waist, with the front section being positioned at or close to the wearer's belly, the back section being positioned against the wearer's lower back, and the crotch section being positioned over the wearer's pubic area and a predefined part of the buttocks, transversally extending between two leg openings formed by the outer cover, such that the garment portion defines an inward facing side intended to face towards the wearer, and an outward facing side, intended to face away from the wearer, when the outer cover is worn,
wherein,
in at least one of the front section and the back section, the outer cover comprises at least one flap member extending from said garment portion, each flap member having a use position wherein an outward facing inner side of the flap member is facing the inward facing side of the garment portion so as to form a space between the outward facing inner side of the flap member and the inward facing side of the garment portion, which space is open in a longitudinal direction towards the crotch section; and wherein
in said at least one of the front section and the back section, the outer cover comprises a set of one or more insert fastener areas being positioned on said outward facing inner side of the at least one flap member, each insert fastener area in said set comprising a surface fastening material for detachably attaching an insert to said outer cover such that, when an insert is attached to the outer cover in said absorbent article, said one or more insert fastener areas of the at least one flap member allow detachable attachment to the inward facing side of a respective front edge portion or a back edge portion of the insert received in said space, while the insert extends inwardly over at least part of the crotch portion of the garment portion.

98. The outer cover according to claim 97, wherein, in said at least one of the front section and the back section, each flap member is located at a front edge or a back edge, respectively, of the outer cover, and/or wherein in said at least one of the front section and the back section, each flap member is extending from the garment portion at least along a back edge or a front edge, respectively, of the flap member, and/or
in said at least one of the front section and the back section, each flap member has a longitudinal length being no more than 20% of the longitudinal length of the outer cover, and/or
each of the front section and the back section comprises at least one flap member, and a sum of the maximum longitudinal lengths of the flap members in the front section and the back section is no more than 30% of the longitudinal length of the outer cover, and/or
in said at least one of the front section and the back section, each flap member has an area being no more than 20% of the area of the outer cover, and/or
wherein a total area of all the flap members of the outer cover is no more than 30% of the area of the outer cover.

99. The outer cover according to claim 97, wherein, in at least one of the front section and the back section of the outer cover, the at least one flap member is a single flap member.

100. The outer cover according to claim 99, wherein the crotch section of the outer cover defines a minimum transversal width of the outer cover, and said flap member width is no less than the minimum transversal width of the outer cover.

101. The outer cover according to claim 97, wherein in said at least one of the front section and the back section, and in at least one of the one or more insert fastener areas of said set, the surface fastening material is adapted for allowing detachable mechanical and/or frictional fastening thereto by a matching surface fastening material, and/or
in said at least one of the front section and the back section, and in at least one of the one or more insert fastener areas of said set, the surface fastening material comprises a plurality of discrete fastening elements, and/or
in said at least one of the front section and the back section, the outer cover comprises one or more insert fastener members being attached to the outward facing inner side of the respective flap member, each insert fastener member providing at least one out of said one or more insert fastener areas in said set.

102. The outer cover according to claim 97, wherein, in said at least one of the front section and the back section of the outer cover, the set of one or more insert fastener areas spans over a transversal fastening width.

103. The outer cover according to claim 97, wherein the outer cover comprises a pair of leg elastic members, arranged along the leg openings of the garment portion at least in the crotch section thereof.

104. The outer cover according to claim 103, wherein in at least one of the front section and the back section of the outer cover, the transversal fastening width is less than a maximum leg elastics spacing width being the maximum transversal distance between the leg elastic members in the outer cover.

105. The outer cover according to claim 97, wherein in at least one of the front section and the back section, the set of one or more insert fastener areas comprises a first subset of one or more insert fastener areas and a second subset of one or more insert fastener areas, wherein the first subset and the second subset are spaced by a transversal spacing width.

106. The outer cover according to claim 105, wherein the transversal spacing width is no less than 40% of the transversal fastening width.

107. The outer cover according to claim 97, wherein at least 50% a total area of the outer cover is liquid permeable and/or wherein at least 50% of a total area of the outer cover is breathable.

108. The outer cover according to claim 97, wherein the garment portion comprises one or more fabric panels.

109. An absorbent insert for use with an outer cover for forming an absorbent article adapted to be worn about a wearer's waist, the insert being shaped to, in use, cover a wearer's pubic area and a predefined part of the buttocks, and the insert extending along a longitudinal direction, and along a transversal direction, which is perpendicular to the longitudinal direction, wherein a thickness direction is defined perpendicular to the transversal and longitudinal directions, said thickness direction being an inward direction, towards the wearer, when the absorbent article is worn, and an outward direction, away from the wearer, when the article is worn,
wherein
the insert has a longitudinal length that extends along the longitudinal direction and the insert is subdivided into a front section, a back section and a crotch section along said longitudinal length,
the insert comprises a liquid impermeable backsheet, a liquid permeable topsheet, and an absorbent assembly between said backsheet and said topsheet, and
the insert has an inward facing side comprising said topsheet to be directed towards the wearer, and an outward facing side to be directed towards the outer cover, when the article is in use, wherein
in at least one of the front section and the back section of the insert, the insert comprises a set of one or more cover fastener areas being provided on the inward facing side of the insert, each cover fastener area comprising a surface fastening material for detachably attaching said insert to said outer cover.

110. The absorbent insert according to claim 109, wherein the insert extends longitudinally between a front edge and a back edge, and wherein, in said at least one of the front section and the back section of the insert, the set of one or more cover fastener areas is positioned at or adjacent to the respective front or back edge of the insert, and/or in said at least one of the front section and the back section, and in at least one of the one or more cover fastener areas in said set, the surface fastening material is adapted for allowing detachable mechanical and/or frictional fastening thereto by a matching surface fastening material.

111. The absorbent insert according to claim 109, wherein said at least one of the front section and the back section of the insert comprises one or more cover fastener members being attached at the inward facing side of the insert, each cover fastener member providing at least one out of said one or more cover fastener areas in said set.

112. The absorbent insert according to claim 109, wherein, in said at least one of the front section and the back section of the insert, the set of one or more cover fastener areas spans a longitudinal length, which is no more than 20% of the longitudinal length of the insert, and/or
a sum of a longitudinal length spanned by any one or more cover fastener areas in the front section and a longitudinal length spanned by any one of more cover fastener areas in the back section of the insert is no more than 30% of the longitudinal length of the insert, and/or
in said at least one of the front section and the back section of the insert, each of the one or more cover fastener areas in said set has an area being no more than 10% of the area of the insert, and/or
wherein a total area of all of the one or more cover fastener areas of the insert is no more than 25% of the area of the insert.

113. The absorbent insert according to claim 109, wherein, in at least one of the front and back section, a transversal fastening width spanned by the set of one or more cover fastener areas is at least 70% of the maximum width of the insert in said front or back section, respectively, and/or
the insert has a minimum transversal width in the crotch section of the insert and, in said at least one of the front and back section, a transversal fastening width spanned by the set of one or more cover fastener areas is no less than the minimum transversal width.

114. The insert according to claim 109, wherein the insert extends longitudinally between a front edge and a back edge, and comprises longitudinal side edges connecting the front edge and the back edge, and the insert comprises a pair of insert leg elastic members extending essentially along the longitudinal direction of the insert, and arranged on both sides of the insert, between the absorbent assembly and the longitudinal side edges of the insert, wherein in each one of the front section and the back section of the insert a leg elastics width is defined as the maximum transversal width over said leg elastic members in said front or back section, respectively.

115. The insert according to claim 109, wherein, in said at least one of the back section and the front section, the insert comprises a pair of leg edge portions, each leg edge portion extending longitudinally along a respective leg elastic member, and transversally between said leg elastic member and a respective longitudinal side edge of the insert so as to define a maximum transversal leg edge width between said leg elastic member and said respective longitudinal side edge, the maximum transversal leg edge width being at least 1 cm, and/or
each of the insert leg elastic members of the pair of leg elastic members is sandwiched between an innermost layer of the insert and the backsheet, and/or
an insert leg elastics length is defined as the longitudinal length of the insert along which the insert leg elastic members are active, said insert leg elastics length being at least 50% of a total longitudinal length of the insert, and/or
in said at least one of the front section and the back section of the insert, the set of one or more cover fastener areas is confined to a longitudinal section of the insert not spanned by the leg elastics length of the pair of insert leg elastic members.

116. The absorbent insert according to claim 109, wherein the inward facing side of the insert comprises a pair of inner side leakage barriers, extending in the longitudinal direction of the insert.

117. The absorbent insert according claim 109, wherein, in said at least one of the back section and the front section, said set of one or more cover fastener areas comprises a first subset of one or more cover fastener areas and a second subset of one or more cover fastener areas, wherein the first and second subsets are spaced.

118. The absorbent insert according to claim 109, wherein the absorbent assembly is positioned at least in the crotch section of the insert and has an absorbent assembly length that spans no more than 80% of the longitudinal length of the insert.

119. The absorbent insert according to claim 109, wherein the backsheet is breathable.

120. The absorbent insert according to claim 109, wherein the absorbent assembly comprises super absorbent polymers.
